(19) United States
(12) Patent Application Publication
(10) Pub. No.: US 2026/0055081 A1
(43) Pub. Date: Feb. 26, 2026

(54) Metal Chelator Compounds, Coordination Complexes, and Methods of Preparation and Use Thereof

(71) Applicant: University of Puerto Rico, San Juan, PR (US)

(72) Inventors: Arthur Tinoco, Guaynabo, PR (US); Israel Rodriguez, Humacao, PR (US); Christopher Nieves, Carolina, PR (US)

(21) Appl. No.: 19/105,503

(22) PCT Filed: Aug. 23, 2023
(86) PCT No.: PCT/US2023/030959

§ 371 Date: Feb. 21, 2025

Related U.S. Application Data
(60) Provisional application No. 63/400,634, filed on Aug. 24, 2022.

(51) Int. Cl. C07D 401/12; A61K 31/4439; A61P 35/02

(52) U.S. Cl. CPC C07D 401/12; A61K 31/4439; A61P 35/02

(57) ABSTRACT

Metal chelation compounds and associated coordination complexes are provided. In one aspect, this disclosure provides for a metal chelation compound, the compound having the general structure: D-L-T, wherein D is deferasirox or a derivative thereof, T is triapine or a derivative thereof, and L is a linking group and is a bond, C₁-C₁₀ alkyl, C₂-C₁₀ alkenyl, or —(C₀-C₃ alkyl)-R—(C₀-C₃ alkyl)-, wherein R is aryl, cycloalkyl, or polyethylene glycol.

### CROSS-REFERENCE TO RELATED APPLICATIONS

This application claims the benefit of U.S. Provisional Application No. 63/400,634, filed Aug. 24, 2022, the disclosure of which is incorporated by reference herein in its entirety.

### STATEMENT REGARDING FEDERALLY SPONSORED RESEARCH

The claimed invention was made with U.S. Government support under grant number 1R21CA240997-01A1 awarded by the National Institutes of Health (NIH). The government has certain rights in this invention.

### FIELD

This disclosure relates to metal chelator compounds, including the formed metal coordination complexes. The disclosure also provides methods for preparation and use thereof, such as for treatment of cancer.

### BACKGROUND

As a consequence of the COVID-19 pandemic, the burden of cancer incidence and mortality is expected to rise so sharply as to cause a cancer pandemic (Sung et al., 2021, Global cancer statistics 2020: GLOBOCAN estimates of incidence and mortality worldwide for 36 cancers in 185 countries. CA: Cancer J. Clin. 71 (3): 209-249). In 2020 alone, 10 million people died from cancer and another 20 million cancer cases were diagnosed across the world. In response to this crisis, many anticancer research efforts are directed at targeting the hallmarks of cancer, the different pathways enabling cell growth, proliferation, and metastasis (Hanahan, 2022, Hallmarks of cancer: New dimensions. Cancer Discov. 12 (1): 31-46). Essential metals like iron (Fe) underly these cancer hallmarks, inter alia because cancer cells heavily rely on Fe in order to multiply and divide at the rapid rate that they do (Chen et al., 2019, Iron metabolism and its contribution to cancer (Review). Int. J Oncol. 54 (4): 1143-1154). They have far higher levels of serum transferrin (sTf) receptors than healthy cells do, indicating that they have a higher dependence on Fe, the metal that sTf transports from the blood into the cell (Shen et al., 2018, Transferrin receptor 1 in cancer: a new sight for cancer therapy. Am. J. Cancer Res. 8(6): 916-931). An increase in Fe influx is coupled with a decrease in its efflux due to decreased levels of Fe exporters (Bystrom et al., 2015, Cancer cells with irons in the fire. Free Radic. Biol. Med 79: 337-42). This, in turn, results in cancer cells having elevated levels of the intracellular labile Fe pool (LIP), necessary to metalate Fe-dependent enzymes/proteins for rapid cell division (Lane et al., 2015, Cellular iron uptake, trafficking and metabolism: Key molecules and mechanisms and their roles in disease. Biochim. Biophys. Acta 1853 (5): 1130-44). Human ribonucleotide reductase (RNR), the rate-limiting enzyme of DNA synthesis, requires Fe in order to catalyze the formation of deoxyribonucleotides from ribonucleotides (Greene et al., 2020, Ribonucleotide reductases: Structure, chemistry, and metabolism suggest new therapeutic targets. Annu. Rev. Biochem. 89: 45-75) and it is overexpressed in cancer cells. An important mode of attacking the viability of cancer cells is targeting Fe bioavailability via the use of Fe-chelating ligands (Yu et al., 2012, Iron chelators for the treatment of cancer. Curr. Med. Chem. 19(17): 2689-2702; Lui et al., 2015, Targeting cancer by binding iron: Dissecting cellular signaling pathways. Oncotarget 6 (22): 18748-18779). For this reason, chelator drugs designed to treat iron overload diseases like the FDA-approved deferasirox (brand name Exjade) are being repurposed to explore their anticancer potential (Szlasa, et al., 2022, Iron chelates in the anticancer therapy. Chem. Pap. 76 (3): 1285-1294). Other chelator drugs, such as triapine (Liu et al., 1992, Synthesis and antitumor activity of amino derivatives of pyridine-2-carboxaldehyde those micarbazone. J Med Chem. 35 (20): 3672-3677), are designed to be used as anticancer agents (Finch, et al., 2000, Triapine (3-aminopyridine-2-carboxaldehyde-thiosemicarbazone): a potent inhibitor of ribonucleotide reductase activity with broad spectrum antitumor activity. Biochem. Pharmacol. 59 (8): 983-91; Shao et al., 2006, Ribonucleotide reductase inhibitors and future drug design. Curr. Cancer Drug Targets 6 (5): 409-31; Shao et al., 2006, A Ferrous-Triapine complex mediates formation of reactive oxygen species that inactivate human ribonucleotide reductase. Mol. Cancer Ther. 5 (3): 586-92). While some chelators can operate extracellularly by blocking cellular Fe uptake, others work intracellularly by scavenging cytosolic Fe from the LIP and disrupting the activation of Fe-dependent enzymes and also triggering Fe-centric toxicity such as ferroptosis (Grignano et al., 2020, From iron chelation to overload as a therapeutic strategy to induce ferroptosis in leukemic cells. Front. Oncol. 10: Front. Oncol. 10: 586530; Li & Zhang, 2022, From iron chelation to overload as a therapeutic strategy to induce ferroptosis in hematologic malignancies. Hematology 27 (1): 1163-1170).

Accordingly, there remains a need to develop new compounds for use as therapeutic agents directed at iron, iron metabolism, and iron uptake and utilization in cancer cells.

### SUMMARY

This disclosure provides metal chelation compounds, the compounds having the structure D-L-T, wherein
D is deferasirox or a derivative thereof,
T is triapine or a derivative thereof, and
L is a linking group and is a bond, C₁-C₁₀ alkyl, C₂-C₁₀ alkenyl, or —(C₀-C₃ alkyl)-R—(C₀-C₃ alkyl)-, wherein R is aryl, cycloalkyl, or polyethylene glycol.

The invention also provide coordination complexes, wherein the coordination complex comprises at least one of the metal chelation compound as otherwise described herein coordinated to at least one metal ion, wherein each metal ion is independently iron, titanium, platinum, vanadium, copper, or gold.

Further provided are methods for preparing metal coordination compounds, the methods comprising:
providing a triapine derivative of structure

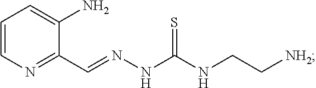 [SMILES: NCCNC(=S)N/N=C/c1ncccc1N]

and
contacting the triapine derivative with deferasirox in an anhydrous polar solvent in the presence of base and hydrobenzotriazole to form a crude mixture.

Also provided are methods for preparing coordination complexes, the methods comprising:
providing the metal chelation compound as otherwise described herein, and
contacting the metal chelation compound with a metal salt in a solvent, wherein the metal of the metal salt is iron, platinum, titanium, vanadium, copper, or gold.

The invention advantageously also provides methods for treating cancer in a subject in need thereof, the methods comprising administering a therapeutically effective amount of the compound as otherwise described herein or the coordination complex as otherwise described herein.

Within this disclose are kits for the treatment of cancer, comprising the compound as otherwise described herein or the coordination complex as otherwise described herein.

Other aspects of the disclosure will be apparent to those skilled in the art in view of the description that follows.

### BRIEF DESCRIPTION OF THE DRAWINGS

FIG. 1 is a diagram describing the synthesis and targeting approach accordingly to an example embodiment.

FIGS. 2A-2B are graphs characterizing in situ Fe(III) DefNEtTrp complexation. FIG. 2A shows the results of MALDI-TOF MS analysis (positive ion mode) of Fe₃(DefNetTrp)₂ ([DefNEtTrp]=100 μM): m/z=1348.10, {C₆₀H₄₈N₁₈O₆S₂Fe₃⁺ (in the MALDI-TOF source, two of the Fe ions in this species are reduced to Fe(II)). FIG. 2B shows UV-Vis spectra of Def, Trp, DefNEtTrp, Fe(Def)₂, Fe(Trp)₂, and Fe₃(DefNEtTrp)₂. The concentration of the ligands in these spectra was maintained at 50 μM. The inset is a zoom-in of the spectra of Fe(III) compounds. FIG. 2C is a cyclic voltammogram of 6 mM Fe₃(DefNEtTrp)₂.

FIGS. 3A-3C show the results of MALDI-TOF MS (positive ion mode) of the metal DefNEtTrp compounds. In FIG. 3A. m/z=1253.35, {(Na⁺)[C₆₀H₅₀N₁₈O₆S₂Ti(IV)]}⁺. In FIG. 3B. m/z=1713.15, {(Na⁺)[C₆₀H₄₈N₁₈O₆S₂Ti(IV)Pt(II)₂Cl₂]}⁺. In FIG. 3C. m/z=1691.15, {[C₆₀H₄₂N₁₈O₆S₂Ti(TV)Au(II)₂Cl₂]}.

FIG. 4A shows UV-Vis spectra of DefNEtTrp (i), Ti(DefNEtTrp)₂ (ii), Ti(DefNEtTrp-Pt(II)-Cl)₂ (iii) and Ti(DefNEtTrp-Au(III)-Cl)₂ (iv). The DefNEtTrp concentration was maintained at 50 μM. FIG. 4B shows UV-Vis difference spectrum of 25 μM Ti(DefNEtTrp)₂ (v) compared with the UV-Vis spectrum of 25 μM Ti(Def)₂ (vi). FIG. 4C shows UV-Vis difference spectra of Ti(DefNEtTrp-Pt(II)-Cl)₂ (vii) and Ti(DefNEtTrp-Au(III)-Cl)₂ (viii) both at 25 μM.

FIG. 5 is a bar graph showing observed changes in the % cell viability of Jurkat cells treated with 10 μM Q-VD-OPh (Q-V-O) and then treated for 72 h with 2 NM DefNetTrp (DNT), 2 μM cisplatin (Cis), or media alone. * p-value <0.01 vs the corresponding non-treated Q-V-O group.

FIG. 6 is a bar graph showing observed changes in the % cell viability of Jurkat cells treated with 10 μM Ferrostatin-1 (Fer-1) and then treated for 72 h with 2 μM DefNetTrp (DNT), M Fe(Citrate)₂ (Fe), or media alone. * p-value <0.01 vs the corresponding non-treated Fer-1 group.

FIG. 7 is an ellipsoid plot of NEtTrp·2HCl (4). The thermal ellipsoids are drawn at 50% probability level. Selected bond lengths (A) and torsion angles (deg): C6-N3 1.275(3), N3-N4 1.357(2), N4-C7 1.357(3), C7-S 11.682(2), C7-N5 1.331(3), N5-C8 1.448(3), C8-C9 1.508(3), C9-N6 1.480(3) Å; Θ_{(N1-C1-C5-C6)} −5.8(3°), Θ_{(C5-C6-N3-N4)} 178.3(2°), Θ_{(N3-N4-C7-S1)} 179.7(1°), Θ_{(N3-N4-C7-N5)} −1.0(3)°, Θ_{(N5-C8-C9-N6)} −69.5(2)°

FIG. 8 is a ¹H NMR spectrum of methyl-N-(2-tert-butoxycarbonyl aminoethyl) dithio carbonate (2). [Conditions: 500 MHz, DMSO-d6].

FIGS. 9A-9B are ¹H NMR spectrum of t-butyl(2-hydrazinecarbothioamide ethyl)carbamate (3). [Conditions: 500 MHz, DMSO-d6]. FIG. 9A is the entire spectrum, and FIG. 9B is the zoom-in of the 1.0-4.5 ppm region.

FIGS. 10A-10B are ¹H NMR spectrum of NEtTrp·2HCl (4). [Conditions: 500 MHz, DMSO-d6]. FIG. 10A is the entire spectrum, and FIG. 10B is the zoom-in of the 7.0-8.5 ppm region.

FIG. 11 is a graph showing MALDI-TOF MS (positive) analysis, wherein m/z 594.23, {(H⁺)[C₃₀H₂₇N₉O₃S]}⁺ (H⁺ adduct of DefNEtTrp (6)).

FIGS. 12A-12B are ¹H NMR spectrum of DefNEtTrp (6). [Conditions: 500 MHz, DMSO-d6]. FIG. 12A is the entire spectrum, and FIG. 12B is the zoom-in of the 3.2-8.6 ppm region.

FIGS. 13A-13B are ¹³C NMR spectrum of DefNEtTrp (6). [Conditions: 500 MHz, DMSO-do]. FIG. 13A is the entire spectrum, and FIG. 13B is the zoom-in of the 115-170 ppm region.

FIG. 14 is a graph showing pH dependent aqueous speciation model for the interaction of 50 μM Fe(III) and fully deprotonated Def- and Trp at 100 μM each. This model only takes homoleptic compounds into consideration because that is the only speciation data available: (i) [Fe(HDef)]⁺; (ii) [Fe(Def)]; (iii) [Fe(HDef)₂]⁻; (iv) [Fe(HDef)(Def)]₂⁻; (v) [Fe(Def)₂]³⁻; (vi) [Fe(HTrp)₂]⁺; and (vii) [Fe(Trp)]²⁺.

FIG. 15 shows MALDI-TOF MS (positive ion mode) analysis wherein m/z 1240.41, {(H⁺)₂[C₆₀H₅₀N₁₈O₆S₂Fe]}⁺ (Fe(DefNetTrp)₂); m/z 1293.32, {C₆₀H₄₉N₁₈O₆S₂Fe₂}⁺ ((Fe₂(DefNetTrp)₂); m/z=1348.10, {C₆₀H₄₈N₁₈O₆S₂Fe₃}⁺ (Fe₃(DefNetTrp)₂) (7) (in the MALDI TOF source, two of the Fe ions in this species are reduced to Fe(II)). The measured data is in black, and the theoretical overlays for Fe(DefNEtTrp)₂, Fe₂(DefNEtTrp)₂, and Fe₃DefNEtTrp)₂ are in green, orange, and red, respectively. The proposed structures for Fe(DefNEtTrp)₂, Fe₂(DefNEtTrp)₂, and Fe₃(DefNEtTrp)₂ are shown left to right. Note that ligand L is not detected by the MALDI-TOF instrument.

FIG. 16 is a graph showing cyclic voltammogram of 6 mM Fe(Def)₂

FIG. 17 shows UV-Vis spectra of triapine (Trp) (solid lines) and Ti(IV)-Trp system (dash lines) recorded at different pH values. ([ligand]=50 μM, metal ligand ratio 1:2; t=25.0° C., I=0.10 M (NaCl) in 50% (w/w) ethanol/water).

FIGS. 18A-18C show proposed structures for Ti(DcfNEtTrp)₂ (8), Ti(DefNEtTrp-Pt(II)-Cl)₂ (9) and Ti(DefNEtTrp-Au(III)-Cl)₂ (10).

FIGS. 19A-19I show dose response curves of DefNEtTrp (6) against the nine different panels of cancer cell lines in the NCI-60 five dose screen.

FIG. 20 shows dose response curves for DefNEtTrp, NEtTrp, Def, Trp, 1:1 combination of Def and NEtTrp, 1:1 combination Def and Trp, against the Jurkat Leukemia cell line for 72 h.

FIG. 21 is a bar graph showing observed changes in the % cell viability of Jurkat cells presupplemented with 2 μM and 5 μM Fe(III)(Citrate)₂ (pH 7.4) and then treated for 72 h with 2 μM DefNetTrp (DNT) or media alone. * p-value <0.01 vs the corresponding non-supplemented Fe group.

FIG. 22 is a dose response curve for DefNEtTrp against the MRC-5 noncancer lung cell line for 72 h.

FIG. 23 is a bar graph showing observed changes in the % cell viability of Jurkat cells treated with 10 μM Ferrastatin-1/Q-VD-OPh (Fer-1/Q-V-O) in combination and then treated for 72 h with 2 μM DefNetTrp (DNT), or media alone. * p-value <0.01 vs the corresponding non-treated Fer-1/Q-V-O group.

FIG. 24 shows a crystal and structure refinement for NEtTrp·2HCl (4).

FIG. 25 is a table showing a summary of the NCI 60 cancer cell line viability screen of DefNEtTrp (6) at the five-dose level for 48 h.

FIG. 26 is a table showing an evaluation of the hemolytic activity of the compounds Trp, NEtTrp, Def, and DefNEtTrp. Positive control: melittin. Dₘₐₓ is the maximal percentage of hemolysis measured.

### DETAILED DESCRIPTION

Targeting iron metabolism has emerged as a novel therapeutic strategy for the treatment of cancer. As part of these efforts, iron chelator drugs are repurposed or are specifically designed as anticancer agents. Two important chelators deferasirox (Def) and triapine (Trp) are understood to attack the intracellular supply of iron (Fe) and inhibit Fe-dependent pathways responsible for cellular proliferation and metastasis. Trp, in particular, forms a redox active ferrous complex that inactivates the Fe-dependent enzyme ribonucleotide reductase (RNR), responsible for DNA replication. Building on recent efforts to synergize intracellular Fe chelation with cytotoxic metals for anticancer therapy, the Fe dual chelator ligand DefNEtTrp, consisting of the Def and Trp moieties, was developed to exploit their respective hard and soft Lewis basicity for effective Fe(II/III) binding and redox modulation and to facilitate formation of heterobimetal complexes. Using UV-Vis spectroscopy, MALDI-TOF mass spectrometry, and cyclic voltammetry analyses, DefNEtTrp is shown herein to generate a redox active Fe(III) complex Fe₃(DefNEtTrp)₂ featuring a reduction potential within the biological window to quench the RNR activating tyrosyl radical. The ligand was also used to synthesize the heterobimetallic complexes Ti(DefNEtTrp-Pt(TT)-Cl)₂ and Ti(DefNEtTrp-Au(Tl)-Cl)₂. Proposed coordination structures were evaluated for insight into the optimal combination of metal ions for preparing specific heterobimetal compounds. Screened against different cancer cell line types, DefNEtTrp exhibited potent and broad spectrum antiproliferative behavior. As seen in Jurkat cell viability experiments set forth below, the cytotoxicity of DefNEtTrp was superior to that of unconjugated Def and Trp ligands in single-drug and combination drug treatments, and was assessed in the context of intracellular labile Fe binding.

Recent work has focused on a transmetalation approach to target intracellular Fe (Parks et al., 2014, Applying the Fe(III) binding property of a chemical transferrin mimetic to Ti(IV) anticancer drug design. Inorg. Chem. 53 (3): 1743-1749; Loza-Rosas et al., 2017, Expanding the therapeutic potential of the iron chelator deferasirox in the development of aqueous stable Ti(IV) anticancer complexes. Inorg. Chem. 56 (14): 7788-7802). Titanium(IV) (Ti(IV)) complexes with high affinity Fe(III) chelators as ligands were developed to synergize intracellular Fe chelation with the cytotoxic potential of Ti(IV). These complexes would undergo induced dissociation by transmetalating the Ti(IV) for Fe(III) and are expected to operate only in the intracellular environment by reacting with the LIP (Gaur et al., 2021, Iron chelator transmetalative approach to inhibit human ribonucleotide reductase. JACS Au 1 (6): 865-878). The Ti(deferasirox)₂ complex exhibited RNR inhibitory capability contributed by both the Ti(IV) and the chelator. Ti(IV) was capable of decreasing the nucleotide substrate pool by coordinating thereto and even cleaving them by phosphate hydrolysis. In binding Fe(III), deferasirox (Def also known as DFX) was capable of inhibiting activation of the RNR enzyme at its R2 subunit. Labile Fe(II) binds to a di-iron cluster site in the R2 subunit via oxidative addition and this process generated an important tyrosyl radical located nearby. This tyrosyl radical activated RNR by traveling to the R1 catalytic cysteine group, priming the initiation of ribonucleotide reduction. Def also was capable of forming a possible redox active Fe monoDef species that can lead to the reduction of the RNR R2 tyrosyl radical and inactivate the enzyme. In Jurkat cells, Ti(Def)₂ arrested the cell cycle at the S phase, indicative of suppressed DNA replication likely from RNR inhibition.

This disclosure provides an expansion of the tools available for anticancer drug design centered on synergizing the targeting of intracellular labile Fe and the cellular delivery of cytotoxic metals by enhancing the chelation template to create a dual chelator ligand. This ligand was designed to feature hard and soft Lewis base chelators to facilitate both effective Fe(II)/Fe(III) binding and the development of heterometallic compounds. To this end, a dual chelator consisting of a covalent conjugation of the hard Lewis base ONO chelator Def and of the soft/intermediate Lewis base NNS triapine (Trp) moieties was synthesized (Scheme 1). Triapine was judiciously selected to expand on the already established utility of the Def moiety in this design for two important reasons. The intracellular LIP consisted of an equilibrium between Fe(II) and Fe(III) but Fe(II) species predominated because of the cellular reducing environment (Kakhlon & Cabantchik, 2002, The labile iron pool: characterization, measurement, and participation in cellular processes. Free Radic. Biol. Med. 33 (8): 1037-1046; Kruszewski, 2003, Labile iron pool: the main determinant of cellular response to oxidative stress. Mutat. Res.—Fundam. Mol. Mech. 531 (1-2): 81-92; Cabantchik, 2014, Labile iron in cells and body fluids: physiology, pathology, and pharmacology. Front. Pharmacol. 5: 45). Unlike Def, Trp features both high Fe(II) and Fe(III) affinity at pH 7.4 (cytosolic pH) and thus enabled the dual chelator conjugate to more broadly interact with the LIP (Enyedy et al., 2011, Interaction of triapine and related thiosemicarbazones with iron(III)/(II) and gallium(III): A comparative solution equilibrium study. Dalton Trans. 40 (22): 5895-905). Trp can form the redox active Fe(II)(Trp)₂ species within cells, which is an effective reductant of the RNR R2 tyrosyl radical (Aye et al., 2012, Mechanistic studies of semicarbazone triapine targeting human ribonucleotide reductase in vitro and in mammalian cells: Tyrosyl radical quenching not involving reactive oxygen species. J Biol. Chem. 287 (42): 35768-35778). Molecular docking has shown that Trp can bind near the RNR R2 di-iron cluster site revealing a route by which the Fe(II)(Trp)₂ species may come into close contact with the tyrosyl radical (Popovic-Bijelic et al., 2011, Ribonucleotide reductase inhibition by metal complexes of triapine (3-aminopyridine-2-carboxaldehyde thiosemicarbazone): a combined experimental and theoretical study. J. Inorg. Biochem. 105 (11): 1422-31). It has also been observed to induce ferroptosis by the generation of reactive oxygen species (ROS) (Zhang et al., 2021, Triapine/Ce6-loaded and lactose-decorated nanomicelles provide an effective chemo-photodynamic therapy for hepatocellular carcinoma through a reactive oxygen species-boosting and ferroptosis-inducing mechanism. Chem. Eng. J 425: 131543). As disclosed herein, the dual chelator deferasirox N-ethyleneamine triapine (DefNEtTrp) was synthesized and its in situ Fe(III) complexation and heterometal compound formation explored with a combination of hard and soft Lewis acidic metal ions. The Fe binding capability of the dual chelator and its associated redox activity were assessed in the context of its broad-spectrum anticancer activity and highly potent cytotoxicity, superior to the behavior of the unconjugated chelators in individual and combined treatments against Jurkat cells.

In one aspect, disclosed herein are metal chelation compounds, the compounds having the structure D-L-T, wherein
D is deferasirox or a derivative thereof,
T is triapine or a derivative thereof, and
L is a linking group and is a bond, C₁-C₁₀ alkyl, C₂-C₁₀ alkenyl, or —(C₀-C₃ alkyl)-R—(C₀-C₃ alkyl)-, wherein R is aryl, cycloalkyl, or polyethylene glycol.

For example, in certain embodiments, the metal chelation compound is provided in the absence of a metal, e.g., in the absence of a transition metal.

Provided as part of this disclosure are suitable derivatives of defeasirox and/or triapine include salts, including pharmaceutically acceptable salts, and the products of chemical derivation including esterification, alkoxylation, methylation (e.g., N-methylation), and the formation of amides. For example, in certain embodiments as otherwise described herein, D is an amide derivative of deferasirox, or a salt thereof. In particular embodiments, D is

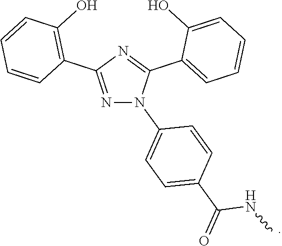

In certain embodiments as otherwise described herein, T is

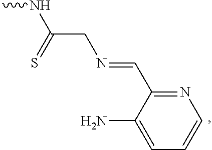

or a salt thereof.

In certain embodiments as otherwise described herein, L is C₁-C₈ alkyl, C₂-C₈ alkenyl, or —(C₀-C₃ alkyl)-R—(C₀-C₃ alkyl)-, wherein R is phenyl, or polyethylene glycol, wherein the polyethylene glycol comprises 1 to 5 ethylene units. For example, in particular embodiments, L is a C₁-C₄ alkyl group, e.g., is —CH₂—CH₂—.

In various embodiments as otherwise described herein, the compound is

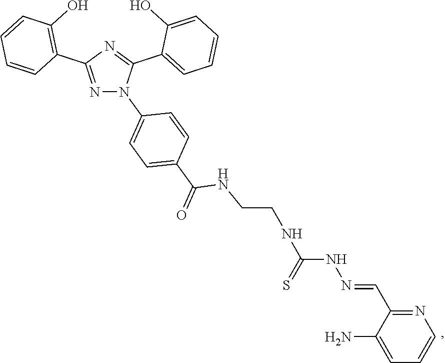 [SMILES: Nc1cccnc1/C=N/NC(=S)NCCNC(=O)c1ccc(-n2nc(-c3ccccc3O)nc2-c2ccccc2O)cc1]

or a salt thereof.

Further disclosed are coordination complexes, wherein the coordination complex comprises at least one of the metal chelation compound as otherwise described herein coordinated to at least one metal ion, wherein each metal ion is independently iron, titanium, platinum, vanadium, copper, or gold. In particular embodiments, the coordination complex comprises two metal chelation compounds that come together to coordinate one metal ion. For example, two D groups may together coordination a single metal ion. Accordingly, in certain embodiments as otherwise described herein, complex has the structure (T-L-D)-M-(D-L-T), M-(T-L-D)-M-(D-L-T), or M-(T-L-D)-M-(D-L-T)-M, wherein M is a metal ion, wherein each M is independently iron, titanium, platinum, vanadium, copper, or gold. For example, in particular embodiments, the complex has the structure M-(T-L-D)-M-(D-L-T)-M. In particular embodiments, the -D-M-D- moiety forms a six-coordinate sphere around the central M. In some embodiments, the coordination spheres of the metal may be completed by anions or solvent molecules. In certain embodiments as otherwise described herein, each metal ion is titanium. In other embodiments, each metal ion is iron, for example, Fe(III). In some embodiments, the complex comprises a mixture of iron and titanium metal ions.

In another aspect, disclosed herein are methods for preparing metal coordination compounds, the methods comprising:
providing a triapine derivative of structure:

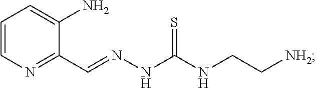 [SMILES: NCCNC(=S)N/N=C/c1ncccc1N]

and
contacting the triapine derivative with deferasirox in an anhydrous polar solvent in the presence of base and hydrobenzotriazole to form a crude mixture.

Examples of suitable anhydrous polar solvents are dimethylformamide, methanol, acetonitrile, and dimethylsulfoxide. Suitable bases include amines, such as pyridine, lutidine, or tertiary amines such as trimethylamine.

In various embodiments as otherwise described herein, the method further comprises: contacting the crude mixture with acid to form a precipitate; and isolating the precipitate.

Suitable acids include mineral acids such as hydrochloride acid, sulfuric acid, nitric acid, or phosphoric acid. Organic acids may also be utilized, such as acetic acid or formic acid.

In another aspect, this disclosure provides methods for preparing a coordination complex, the method comprising:
providing the metal chelation compound as otherwise described herein, and
contacting the metal chelation compound with a metal salt in a solvent, wherein the metal of the metal salt is iron, platinum, titanium, vanadium, copper, or gold.

In another aspect, the disclosure provides methods for treating cancer in a subject in need thereof, these methods comprising administering a therapeutically effective amount of the compound as otherwise described herein or the coordination complex as otherwise described herein.

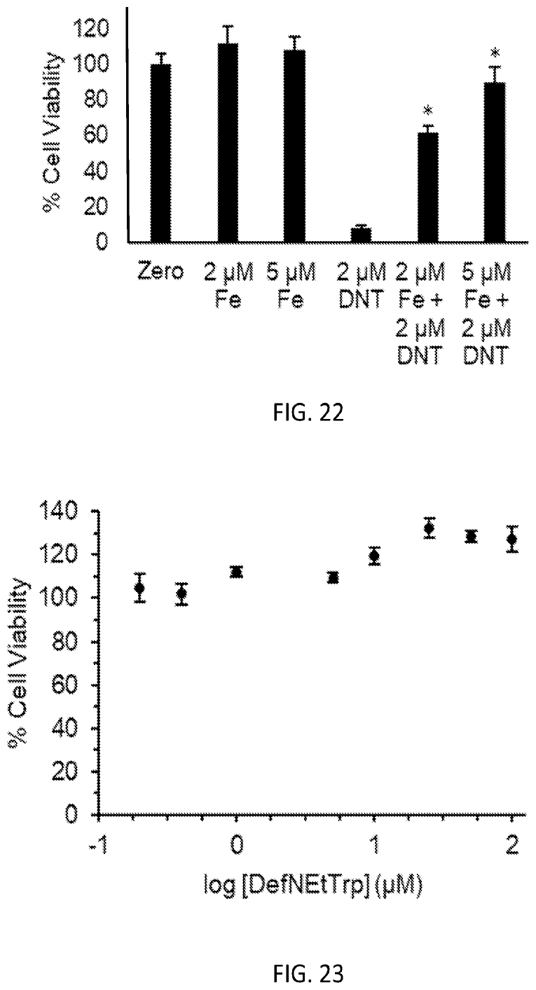

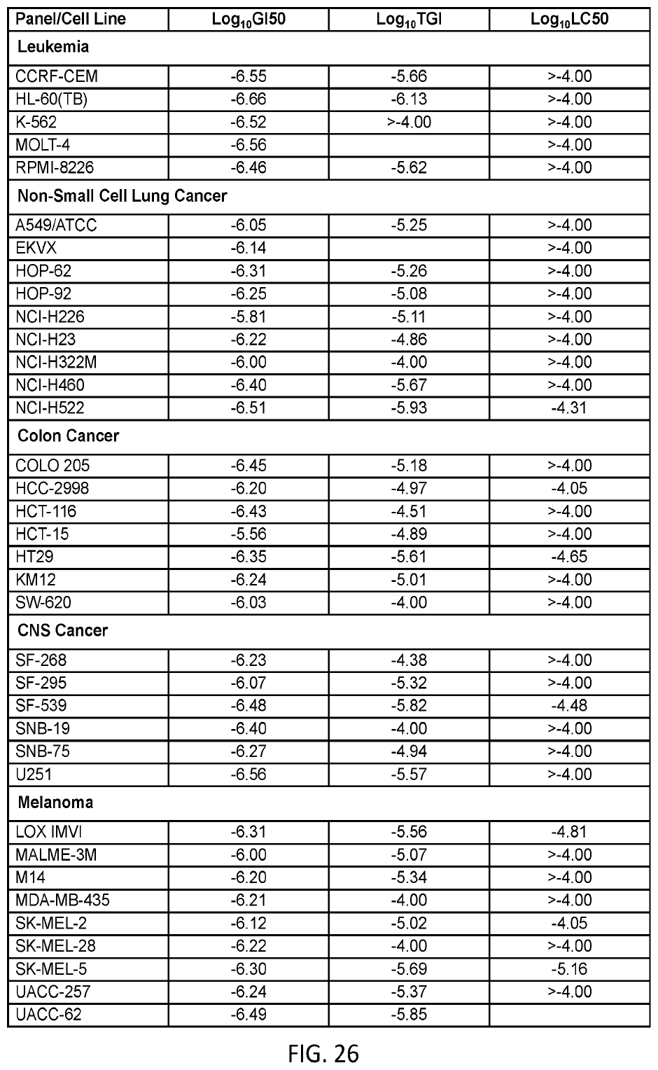

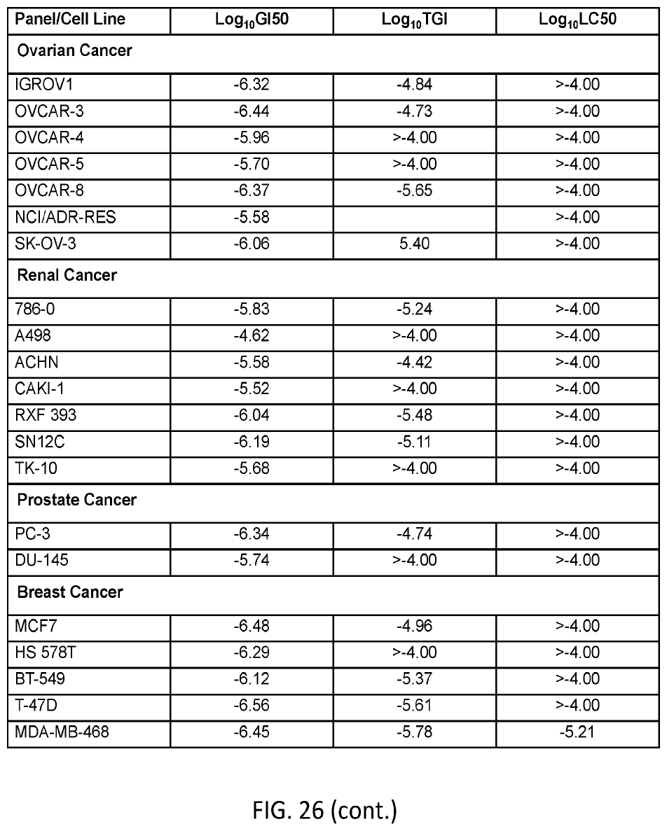

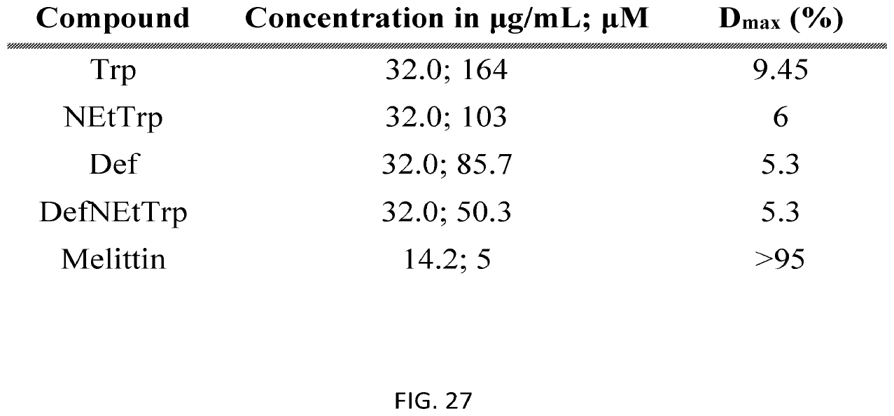

Advantageously, the compounds and the method of this disclosure can be used for highly selective treatment of cancerous cells. As demonstrated in the Example and FIGS. 22 and 26, the compounds of this disclosure can be used as potent antiproliferative agents against cancer cells, while noncancerous cells are largely unaffected. Accordingly, in various embodiments as otherwise described herein, wherein the administering results in an antiproliferative effect against non-cancerous cells of no more than 20%, e.g., no more than 15%, or no more than 10%, or no more than 10%, or no more than 5%.

In another aspect are disclosed kits for treating cancer, comprising the compound as otherwise described herein or the coordination complex as otherwise described herein.

Examples of suitable disease for use according to this invention include various cancers, including leukemia, lung cancer (e.g., non-small cell lung cancer), colon cancer, central nervous system (CNS) cancer, melanoma, ovarian cancer, renal caner, prostate cancer, or breast cancer.

The compound as otherwise described herein can be administered orally or intravenously in dosage unit formulations containing conventional non-toxic pharmaceutically acceptable carriers, adjuvants and vehicles. The pharmaceutical compositions described herein can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known in the art for the manufacture of pharmaceutical compositions and such compositions can contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques, for example with an enteric coating. In some cases such coatings can be prepared by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Formulations for oral use can also be presented as lozenges.

Aqueous suspensions are also provided containing the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

In various embodiments, the methods of this disclosure involve the administration of an effective dose of the compound or coordination complex as otherwise described herein to a subject in need thereof. In certain embodiments as otherwise described herein, the compound can be administered in an amount ranging 0.1 mg/kg to 400 mg/kg. For example, in certain embodiments, the compound can be administered in an amount ranging from 1 mg/kg to 300 mg/kg. In other embodiments, the compound can be administered in amount ranging from 10 mg/kg to 200 mg/kg.

In certain embodiments as otherwise described herein, the dose of compound as otherwise described herein can be administered one or more times per day, such as one time per day, two times per day, three, four, or six times per day. In certain embodiments as otherwise described herein, compound as otherwise described herein is administered for any suitable period of time. For example, the compound as otherwise described herein can be administered for a period of at least three weeks, or a period of 4-6 weeks, or for a period of at least 4 weeks, 6 weeks, 8 weeks, 12 weeks, or at least 24 weeks.

As used herein, the term “alkyl” includes a saturated hydrocarbon having a designed number of carbon atoms, such as 1 to 10 carbons (i.e., inclusive of 1 and 10), 1 to 8 carbons, 1 to 6 carbons, 1 to 3 carbons, or 1, 2, 3, 4, 5 or 6. Alkyl group may be straight or branched and depending on context, may be a monovalent radical or a divalent radical (i.e., an alkylene group). For example, the moiety “—(C₁-C₆ alkyl)-O—” signifies connection of an oxygen through an alkylene bridge having from 1 to 6 carbons and C₁-C₃ alkyl represents methyl, ethyl, and propyl moieties. Examples of “alkyl” include, for example, methyl, ethyl, propyl, isopropyl, butyl, iso-, sec- and tert-butyl, pentyl, and hexyl.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond. Alkenyl group may be straight or branched and depending on context, may be a monovalent radical or a divalent radical (i.e., an alkenylene group). For example, the moiety “—(C₂-C₆ alkenyl)-O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “aryl” represents an aromatic ring system having a single ring (e.g., phenyl) which is optionally fused to other aromatic hydrocarbon rings or non-aromatic hydrocarbon or heterocycle rings. “Aryl” includes ring systems having multiple condensed rings and in which at least one is carbocyclic and aromatic, (e.g., 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3-dihydrobenzofuranyl and tetrahydroisoquinolinyl. The aryl groups herein are unsubstituted or, when specified as “optionally substituted”, can unless stated otherwise be substituted in one or more substitutable positions with various groups as indicated. In circumstances in which a divalent moiety is required and is stated as being “aryl,” those skilled in the art will understand that the term “aryl” refers to the corresponding divalent moiety, arylene.

The term “cycloalkyl” refers to a non-aromatic carbocyclic ring or ring system, which can be saturated (i.e., a cycloalkyl) or partially unsaturated (i.e., a cycloalkenyl). The cycloalkyl ring optionally fused to or otherwise attached (e.g., bridged systems) to other cycloalkyl rings. Certain examples of cycloalkyl groups present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. The cycloalkyl groups herein are unsubstituted or, when specified as “optionally substituted”, can be substituted in one or more substitutable positions with various groups, as indicated.

As used herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

As used herein, the terms “individual,” “patient,” or “subject” are used interchangeably, refers to any animal, including mammals, preferably humans.

As used herein, the phrase “therapeutically effective amount” or “effective amount” refers to the amount of active compound or pharmaceutical agent that elicits the biological or medicinal response that is being sought in a tissue, system, animal, individual or human by a researcher, veterinarian, medical doctor or other clinician.

In certain embodiments, an effective amount can be an amount suitable for
(i) inhibiting the progression the disease;
(ii) prophylactic use for example, preventing or limiting development of a disease, condition or disorder in an individual who may be predisposed or otherwise at risk to the disease, condition or disorder but does not yet experience or display the pathology or symptomatology of the disease;
(iii) inhibiting the disease; for example, inhibiting a disease, condition or disorder in an individual who is experiencing or displaying the pathology or symptomatology of the disease, condition or disorder;
(iv) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or
(v) eliciting the referenced biological effect.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

### EXAMPLES

The Examples that follow are illustrative of specific embodiments of the disclosure, and various uses thereof. They are set forth for explanatory purposes only, and should not be construed as limiting the scope of the disclosure in any way.

### Experimental

Chemicals and materials. N-boc-ethylenediamine (1), methyl iodide, 3-aminopicolinaldehyde, hydroxybenzotriazole (HOBt), 200 proof ethanol, ethyl acetate (98%), anhydrous dimethylformamide (DMF) (99.8%), hydrochloric acid (HCl) (12 M), sodium hydroxide (NaOH) pellets, molecular sieves, sodium bicarbonate, sodium sulfate, potassium titanium oxide oxalate dihydrate (K₂[TiO(C₂O₆)₂]·2H₂O), iron(ITI) chloride (FeCl₃), potassium tetrachloroaurate(III) (KAuCl₄), potassium tetrachloroplatinate(II) (K₂PtCl₄), and tetrabutylammonium hexafluorophosphate were purchased from Sigma. Carbon disulfide and hydrazine hydrate were purchased from Alfa Aesar. 1-Ethyl-3-(3-dimethylaminopropyl)carbodiimide (EDC) was purchased from Fisher Scientific. Ethylene dichloride and deuterated dimethyl sulfoxide (DMSO-d6) were purchased from Merck Millipore. Triapine (Trp) was purchased from MedChemExpress LLC. Deferasirox (Def) was synthesized by modifying a previously reported method (Steinhauser, S.; Heinz, U.; Bartholomä, M.; Weyhermüller, T.; Nick, H.; Hegetschweiler, K., Complex formation of ICL670 and related ligands with Fe(III) and Fe(II). Eur. J Inorg. Chem. 2004, 2004 (21), 4177-4192). Tricitratoiron(III) ([Fe(Citrate)₂]⁵) was prepared in situ by reacting equimolar amounts of iron(III) citrate (Fe(C₆H₅O₇)) (Sigma), and trisodium citrate (Na₃Citrate) (Thermo Scientific) in solution and adjusting the pH to 7.4. Jurkat cells clone E6-1 was obtained from ATCC (ATCC TIB-152) authenticated with the certificate of analysis. Roswell Park Memorial Institute (RPMI) 1640 medium (Corning, CellGro, REF R8758) containing L-glutamine was purchased from Sigma and supplemented with 10% fetal bovine serum (FBS; HyClone) and 1% of antibiotic solution prepared with 11 mg/mL streptomycin and 7 mg/mL penicillin purchased from Sigma. MRC-5 human lung cells were obtained from ATCC® (CCL-171™) The cell line was cultured in phenol red DMEM (Sigma, D6429) containing 1% glutamine, 4.5 g/mL of glucose, and sodium pyruvate. Media was supplemented with 10% FBS (HyClone) and 1% penicillin-streptomycin at 37° C. in a humidified atmosphere of 5% CO₂ (v/v). Phenol Red-free DMEM (CellGro® REF 17-205-CV) was used during cell viability assay studies. 3-(4,5-Dimethylthiazol-2-yl)-2,5-diphenyltetrazolium bromide (MTT) was purchased from EMD Biosciences Inc. Tris was purchased from Amresco. Dodecyl sulfate sodium salt (SDS, electrophoresis, 98% pure) was obtained from Acros Organics. Trypan blue solution (0.4%) was purchased from Sigma. Phosphate buffer saline (PBS) was prepared in lab (pH=7.2). Corning™ Costar™ 96-Well, Cell Culture-Treated, Flat-Bottom Microplate was used to perform the cell viability assays. Ferrostatin-1, Q-VD-OPh, cisplatin (98%) were purchased from MedChem Express (Monmouth Junction, NJ). Human apo-transferrin was obtained from Sigma (T2036). A Microsep 3K MWCO spin dialyzers were obtained from Pall Corp. All other chemicals/solvents were of high purity and used as received. All aqueous solutions were prepared with autoclaved (121° C. and 18 psi) high-quality nanopure water (18.2 MΩ·cm resistivity at 25° C., PURELAB flex system (ELGA LabWater Corp.). Ar and CO₂ USP gases were supplied by Messer Gas.

Instrumentation. A Nicolet iS50 FTIR Spectrometer (ThermoFisher Scientific, WI) was used to collect FTIR absorbance spectra. Proton (¹H) and carbon (¹³C) nuclear magnetic resonance (NMR) spectra were recorded on a Bruker Avance 500 MHz NMR spectrometer (Billerica, Massachusetts, United States) using deuterated dimethyl sulfoxide (DMSO-d6) as the solvent. Chemical shifts were recorded in units of parts per million (ppm. 6) downfield from the peak of the internal standard. ¹H NMR data are reported as follows: chemical shift (ppm), multiplicity (singlet (s), doublet (d), triplet (t), quartet (q), multiplet (m)), coupling constant in Hz, and integration. ¹³C NMR data are reported by chemical shift (ppm). Mass spectra were obtained using an AB SCIEX 4800 MALDI-TOF/TOF mass spectrometer (Framingham, Massachusetts, United States). Cyclic voltammograms were obtained using a SP-240 potentiostat (Bio-logic Science Instrument, Knoxville, Tennessee, United States). Ultraviolet visible (UV-Vis) spectra were obtained using a Cary 300 UV-Vis spectrophotometer (Agilent Technologies, Santa Clara, California, United States). All pH measurements were performed using a Thermo Fisher Scientific Orion Star A211 and an Orion 9157BNMD electrode. The electrode was calibrated in units of mV, using standard buffer solutions at pH 4.01, 7.00, and 10.01. X-ray diffraction analysis was carried out using a Rigaku XtalLAB SuperNOVA single micro-focus Cu-Kα radiation (λ=1.5417 Å) source equipped with a HyPix3000 X-ray detector in transmission mode operating at 50 kV and 1 mA within the CrystAllisPRO software ver. 1. 171.39.43c (The Woodlands, Texas, United States). Cell viability was determined using the MTT assay. Multiwell plate absorbance was measured in an Infinite M200 PRO Tecan Microplate Reader (Morrisville, North Carolina, United States). Cells were grown in a Revco Elite III RCO5000T-5-ABC incubator purchased from Thermo Fisher Scientific. Cell counting was performed by the Trypan blue method (1:5) using a hemocytometer. The counting and culture viability monitoring were performed using a Nikon Eclipse TS-100 microscope (Melville, New Jersey, United States).

Synthesis of methyl-N-(2-tert-butoxycarbonyl amino ethyl)dithiocarbonate (2). N-boc-ethylenediamine (1) (2.0115 g, 12.23 mmol, 1 eq.) was dissolved in ethanol (70 mL) in a 250 mL round bottom flask equipped with a stir bar. Triethylamine (2.04 mL, 14.68 mmol, 1.2 eq.) was added to the flask and the solution was left to stir for five minutes. Carbon disulfide (887 μL, 14.68 mmol, 1.2 eq.) was added to the flask and left to stir for 90 minutes. Methyl iodide (0.914 mL, 14.86 mmol. 1.2 eq.) was then added to the flask and left to stir overnight. Ethyl acetate (30 mL) was added to the flask, resulting in the formation of white crystals. The supernatant was separated from the precipitate through two rounds of centrifugation (4.4 k rpm, 45 min). Ethyl acetate was used to wash the pellets between the two rounds of centrifugation. The supernatant was concentrated using a rotary evaporator, producing a pale yellow, viscous residue. The residue was washed with hydrogen chloride (20 mL), sodium bicarbonate (20 mL), and water (20 mL). The aqueous layer was removed between each wash. The organic layer was concentrated using the rotary evaporator and high vacuum. Methyl-N-(2-tert-butoxycarbonyl amino ethyl)dithiocarbonate (2) was obtained as a pale yellow, viscous oil (2.5 grams, 82% yield). In this work, % yield was determined after purification based on the calculation: experimental mass/theoretical mass×100%. ¹H NMR (500 MHz, DMSO-d₆): δ=9.80 (s, 1H), 6.90 (d, J=5.3, 1H), 3.61 (q, J=5.5, 6.1, 6.2, 2H), 3.15 (q, J=6.2, 2H), 2.52 (q, J=19.9, 3.7, 1.8, 3H), 1.39 (s, 9H).

Synthesis of tert-butyl (2-hydrazinecarbothioamide ethyl)carbamate (3). Methyl-N-(2-tert-butoxycarbonyl amino ethyl)dithiocarbonate (2) (2.4 g, 9.59 mmol, 1 eq.) was dissolved in ethanol (48 mL) in a 100 mL round bottom flask. Hydrazine hydrate (0.800 mL, 16.3 mmol, 1.7 eq.) was added to the flask and the solution was refluxed at 95° C. overnight. The solvent was removed from the flask using rotary evaporation. Ethyl acetate (25 mL) was added to the flask and the organic layer was washed with water (20 mL) three times. The organic layer was dried over anhydrous sodium sulfate and the solvent was removed using a rotovap. The sample was then concentrated overnight using a high vacuum. The thiosemicarbazide (3) was obtained as a pale, yellow viscous oil (1.7 g, 76% yield). ¹H NMR (500 MHz, DMSO-d₆): δ=8.70 (s, 1H), 7.96 (s, 1H), 6.91 (t, J=5.0, 1H), 4.45 (s, 2H), 3.50 (q, J=5.7, 5.8, 5.9, 2H), 3.07 (q, J=5.7, 5.9, 6.0, 2H), 1.38 (s, 9H).

Synthesis of nitrogen ethyl triapine dihydrochloride (NEtTrp·2HCl) (4). Tert-butyl (2-hydrazinecarbothioamide ethyl)carbamate (3) (1.37 g, 5.83 mmol, 1 eq.) was dissolved in ethanol (27 mL) in a 100 mL two-neck round bottom flask. 3-amino picoaldehyde (0.750 g, 5.83 mmol, 1 eq.) was added to the flask and refluxed at 90° C. for ten minutes. Hydrogen chloride (1.94 mL, 23.32 mmol, 4.0 eq.) was added dropwise to the stirring solution, resulting in a color change from yellow to dark green to brown to red to a cloudy orange. The solution was left to stir overnight. The precipitate was isolated through two rounds of centrifugation (4.4 k rpm, 20 min). Ethanol (10 mL) was used to wash the product between the two rounds of centrifugation. The supernatant was removed, and the product was concentrated under high vacuum overnight. NEt-Triapine·2HCl (4) was obtained as a yellow crystalline solid (1.56 g, 86% yield). MALDI-TOF (positive): m/z 239.06, {(H⁺)[C₉H₁₄N₆S]}⁺. ¹H NMR (500 MHz, DMSO-d₆): δ=12.26 (s, 1H), 9.50 (s, 1H), 8.39 (s, 1H), 8.24 (s, 3H), 8.03 (d, J=5.1, 1H), 7.82 (d, J=8.6, 1H), 7.62 (q, J=2.9, 5.3, 5.4, 1H), 7.23 (s, 2H), 3.87 (m, 2H), 3.10 (q, J=5.5, 5.6, 5.8) 2H). ¹³C NMR (500 MHz, DMSO-d₆): δ=177.68, 145.98, 133.12, 130.63, 130.45, 126.74, 126.34, 41.36, and 37.82. FT-IR (cm⁻¹) 3191, 2921, 2851, 1520, 1472, 1322, 1290, 1222, 1169, 1107, 1036, 962, 856, 792, and 702.

Synthesis of deferasirox nitrogen ethyl triapine (DefNEtTrp) (6). Def (5) (0.540 g, 1.45 mmol, 0.9 eq.) and hydrobenzotriazole (0.155 g, 1.01 mmol, 0.63 eq.) were dissolved in anhydrous DMF (10 mL) with a few molecular sieves in an oven-dried 100 mL round bottom flask. The flask was purged with argon. Triethylamine (200 μL, 1.67 mmol, 1.0 eq.) was added to the flask and the mixture was stirred at 0° C. for ten minutes. A solution of EDC (0.401 g, 2.09 mmol, 1.3 eq.) in anhydrous DMF (5 mL) was added dropwise to the flask. The mixture was stirred for another thirty minutes. A suspension of 4 (0.500 g, 1.61 mmol, 1 eq.) and triethylamine (440 μL, 3.33 mmol, 2 eq.) in anhydrous DMF (5 mL) was added dropwise to the flask. The ice bath and argon source were removed, and the solution was left to stir for 48 hours. Hydrochloric acid (10 drops, 12M) was added to the flask to promote precipitation. The precipitate was isolated through four rounds of centrifugation (4.4 k rpm, 20 min). The product was washed with water (10 mL) between each round. The solid was stored at −80° C. for a few hours before being lyophilized. DefNFtTrp (6) was obtained as a yellow solid (0.845 g, 68% yield). The compound was >95% pure. C,H,N elemental analysis was performed by Atlantic Microlabs (Norcross, GA). Anal. Calcd for C₃₀H₂₇N₉O₃S·2H₂O (Mᵣ=629.70 g/mol): C, 57.22 (57.44); H, 4.96 (5.13); N, 20.02 (19.87). MALDI-TOF (positive): m/z 594.23, {(H⁺)[C₃₀H₂₇N₉O₃S]}. ¹H NMR (500 MHz, DMSO-d6): δ=11.43 (s, 1H), 10.83 (s, 1H), 10.07 (s, 1H), 8.52 (s, 1H), 8.37 (s, 1H), 8.06 (d, J=7.1, 1H), 7.95 (d, J=8.3, 2H), 7.85 (d. J=3.6, 1H), 7.54 (d, J=8.3, 3H), 7.38 (t, J=7.7, 2H), 7.20 (d, J=8.1, 1H), 7.09 (q, J=4.1, 4.2, 1H), 7.01 (m, 3H), 6.87 (d, J=8.2, 1H), 6.55 (s, 2H), 3.76 (s, 2H), 3.55 (s, 2H). ¹³C NMR (500 MHz, DMSO-d6): δ=177.13, 166.51, 160.28, 156.82, 155.73, 152.43, 149.79, 144.40, 140.41, 13774, 134.44, 13331, 132.96, 131.91, 13155, 12882, 127.24, 124.96, 123.66, 12280, 120.17, 119.87, 117.54, 116.62, 114.95, 114.17, 44.83, 39.98. FT-IR (cm⁻¹) 3208, 1608, 1531, 1462, 1297, 1244, 1224, 1145, 1099, and 753.

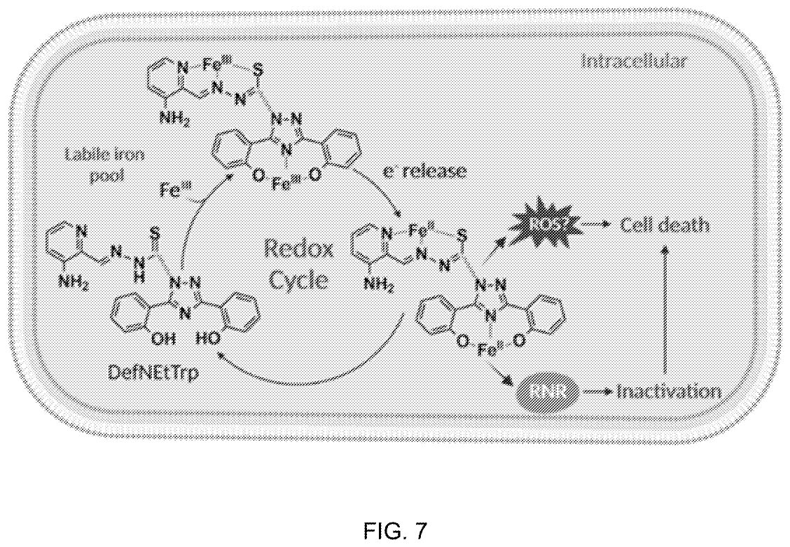

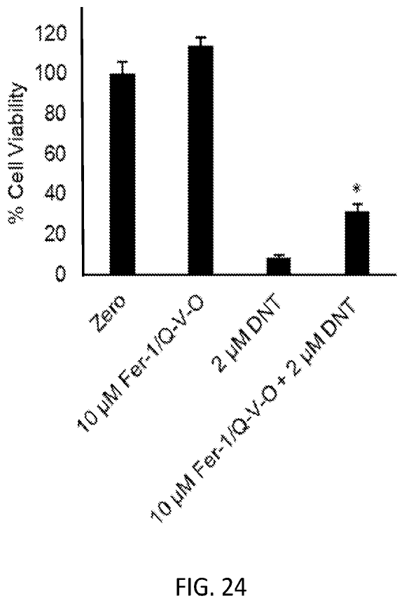

Characterization of nitrogen ethyl triapine dihydrochloride (NEtTrp 2HCl) (4) by Single Crystal X-ray Diffraction. A brown plate-like crystal of NEtTrp-2HCl (4) was mounted on a MiTeGen micro loop for structure elucidation. Structural elucidation was performed using a Rigaku XtalLAB SuperNova single micro-focus Cu-Kα radiation (λ=1.5417 Å) source equipped with a HyPix3000 X-ray detector in transmission mode operating at 50 kV and 1 mA within the CrystAlisPRO software ver. 1.171.39.43c. An Oxford Cryosystems Cryostream 800 cooler controlled the temperature at 293 K. The crystal structure was solved by Intrinsic Phasing using the program ShelXT and refined by full-matrix least squares on F2 using ShelXL within the Olex2 (v1.2-ac3) software. All non-hydrogen atoms were anisotropically refined. All the hydrogen atoms were placed in their calculated positions and then refined using the riding model. Isotropic displacement parameters for these atoms were set to 1.2 times Ueq of the parent atom. A summary of the crystal data, structure solution and refinement is included in the supporting materials (FIGS. 7 and 24). CCDC 2216216 contains the supplementary crystallographic data related to this disclosure, and is incorporated herein in its entirety.

pH dependent speciation model of Fe(III) interaction with Def and Trp. A pH-dependent speciation model for the reaction of 50 μM Fe(111) with 100 μM Def and 100 μM Trp was prepared by using the Species program developed by L. D. Pettit (Academic Software). To generate this model, the pH-dependent formation constants for Fe(III) Def and Fe(III) Trp species, the pKa values of the Def and Trp molecules, the relevant Fe(III) hydrolysis constants, and the pKw of water were used (Stefánsson, A., Iron(III) hydrolysis and solubility at 25° C. Environ. Sci. Technol. 2007, 41 (17), 6117-6123).

Assessing Fe(III) binding by DefNEtTrp. The Fe(III) complex Fe₃(DefNEtTrp)₂ (7) was synthesized in situ by adding a 1 mL solution of FeCl₃ (16.22 mg, 0.1 mmol, 10 eq.) in anhydrous DMF dropwise to a 1 mL stirring solution of DefNEtTrp (6) (5.94 mg, 0.01 mmol, 1 eq.) in anhydrous DMF. The mixture was stirred overnight but was moderately soluble. The compound was examined by MALDI-TOF MS (positive ion mode) at 100 μM concentration following dilution in water to decrease DMF content to 2.1% (v/v). The following proposed ions were observed: m/z=1240.41, {(H⁺)₂[C₆₀H₅₀N₁₈O₆S₂Fe]}⁺ (Fe(DefNetTrp)₂); m/z 1293.32, {C₆₀H₄₉N₁₈NO₆S₂Fe₂}⁺ ((Fe₂(DefNetTrp)₂); m/z 1348.10, {C₆₀H₄₈N₁₈O₆S₂Fe₃}⁺ (Fe₃(DefNetTrp)₂) (in the MALDI-TOF source, two of the Fe ions in this species are reduced to Fe(II)). Confirmation of the species is confirmed by theoretical spectra generation using IsoPro3.1. It is important to note that in a separate synthesis and purification of DefNEtTrp, the ligand was extracted with 6 HCl co-crystal equivalents. For successful Fe₃(DefNEtTrp)₂ synthesis, the HCl co-crystals must be neutralized with NaOH otherwise the reaction solution becomes too acidic to enable Fe(III) coordination. The matrix for all samples examined by MALDI-TOF MS was composed of 5 mg/mL α-Cyano-4-hydroxycinnamic acid and was prepared in a solvent composed of acetonitrile and water in a 1:1 (v/v) ratio plus trifluoroacetic acid at 0.1% (v/v). 1 μL of matrix was placed on the MALDI wells and let to crystalize. Then 1 μL of sample solutions were placed on top of the crystallized matrix and let to crystallize. Mass range was set up from 200 to 2000 m/z. Excel was used to tabulate the data and Isopro3.1 was used to obtain the theoretical spectra of the species identified. The theoretical and experimental intensities were normalized to a maximum signal of 100.

UV-Vis spectra were collected of Fe₃(DefNEtTrp)₂, Fe(Trp)₂, and Fe(Def)₂. The metal complexes were prepared in situ by diluting as required, relatively high concentrations of stock solutions of DefNEtTrp and FeCl₃. A 2 mL Fe₃(DefNEtTrp)₂ solution was prepared by reacting 50 μM of DefNEtTrp with 75 μM of FeCl₃ in 95% DMF and the solution was left to react overnight. Both Fe(Trp)₂ and Fe(Def)₂ solutions (2 mL) were prepared by reacting 50 μM of Trp or Def with 50 μM of FeCl₃ in 95% DMF and left to react overnight.

The reduction potential of 10 mL of 6 mM Fe₃(DefNEtTrp)₂ and Fe(Def)₂ in DMF were examined by cyclic voltammetry (CV). The 10 mL of 6 mM of both compounds were prepared in situ in DMF. NaOH(aq) (1 M) was added to the solutions. The mole amount of NaOH added was based on a separate solution preparation with 50:50 DMF:H₂O that would achieve a raw pH of 7.4 (not solvent corrected). The compound solutions also 0.2 M Nbu₄PF₆ (diluted from a 2M stock prepared in DMF using Nbu₄PF₆ recrystallized from 100% distilled ethanol). Cyclic voltammograms were measured in a three-electrode cell, consisting of a 2.0 mm diameter glassy carbon working electrode, a platinum auxiliary electrode, and an Ag/AgCl reference electrode containing 0.1M KCl in water. All samples were deaerated by passing a stream of argon through them before measurements and then maintaining a blanket of argon over them during measurements. All measurements are reported relative to the normal hydrogen electrode (NHE). Appropriate solution controls were run, blank anhydrous DMF, Def, DefNEtTrp, and FeCl₃ in anhydrous DMF.

The ability of DefNEtTrp to scavenge Fe(ITT) from Fe(ITI)-saturated serum transferrin(Fe₂-sTf) was also examined. Fe₂-sTf was prepared by reacting micromolar amounts of apo-sTf with 4 mol equiv of in situ prepared [Fe(citrate)₂]⁵⁻ in 20 mM Hepes buffer (pH 7.4) containing 0.1 M NaCl and 27 mM NaHCO₃. The sample was extensively dialyzed by rapid spin dialysis to remove the excess unbound Fe(III). The Fe(III) content was determined by the ferrozine colorimetric assay modified from a literature protocol and by monitoring the Fe₂-STf-(CO₃)₂ LMCT band (λₘₐₓ 465 nm; ε=5200 M⁻¹ cm⁻¹) (Viollier 2000, The ferrozine method revisited: Fe(II)/Fe(III) determination in natural waters. Appl. Geochem. 15 (6): 785-790). To 22.5 μM Fe₂-sTf was reacted 45 μM DefNEtTrp (diluted in the same protein buffer while maintaining 5% DMF) in quadruplicate at 25° C. The reaction solutions were left to equilibrate and the LMCT absorbance was monitored after 24 h and 72 h. The reaction solutions were extensively dialyzed and then the protein concentrations were quantified by the Bradford colorimetric assay. The UV-Vis spectra of the washed protein samples were collected.

pH-potentiometric study to explore Trp coordination of Ti(IV). pH-metric measurements were carried out to identify possible complexation between triapine (Trp) with Ti(IV). Stock solutions of the compounds were prepared in pure ethanol to maximize solubility. The ligand were first titrated alone and then in the presence of Ti(IV) at a ratios of 1:2 (Ti(IV):Trp). The stock solutions were diluted to start the titration at 50 μM for Trp in a 50/50 ethanol/water mixture with 0.10 M NaCl at 25.0° C. The pH-metric titrations were performed in the pH range 2.0-12.0. The samples were divided into acid titration and base titration. The initial volume of the samples was 20.0 mL. Aliquots of 1.0 M and 0.1 M HCl or NaOH were titrated into the solution by micropipet in volumes ranging from 2 to 20 μL. After the addition of each aliquot of acid or base, the solutions were equilibrated for ˜10 min, when the millivoltage reading remained constant for 1 min and recorded. A sample of 1 mL was temporarily removed from the mother solution to collect a UV-Vis spectrum (200 nm to 700 nm). The changes in concentration throughout the pH titration were accounted for. The experiments were performed in triplicate.

Assessing the formation of bimetalation compounds of DefNEtTrp with Ti(IV), Au(III), and Pt(II). A solution of K₂[TiO(C₂O₆)₂]·2H₂O (17.9 mg, 0.051 mmol, 2 eq) in 5 mL of water was added drop wise to a stirring solution of DefNEtTrp (6) (16.9 mg, 0.027 mmol, 1 eq) in 5 mL of DMF. After one hour of reaction a yellow precipitate formed. The yellow solid was separated by centrifuge (4.4 k rpm, 20 min) and it was washed with cold water three times and ethanol three times to remove unreacted starting materials. The product was then lyophilized to obtain Ti(DefNEtTrp)₂ (8). A solution of K₂PtCl₄ (8.8 mg, 0.021 mmol. 3 eq) in 5 mL of water was added dropwise to a stirring solution of 8, which was completely dissolved in 5 mL of DMF. The solution was let to react for one hour. The orange-yellow product [TiPt₂(DefNEtTrp)₂]Cl₂ (9) was separated, washed, and isolated similarly to 8. A similar approach was taken to prepare TiAu₂(DefNEtTrp)₂ (10) by adding KAuCl₄ (7.9 mg, 0.021 mmol, 3 eq) in 5 mL of water was added dropwise to a stirring solution of 8 (10 mg, 0.007 mmol, 1 eq) in 5 mL of DMF. The solution was let to react for one hour. The orange-yellow product TiAu₂(DefNEtTrp)₂ (10) was separated, washed, and isolated similarly to 9. The compounds were examined by MALDI-TOF MS (positive ion mode) at 100 μM concentration following dilution in water to decrease DMF content to 5% (v/v). The following proposed ions were observed: 8 m/z 1253.35, {(Na⁺)[C₆₀H₅₀N₁₈O₆S₂Ti]}⁻; 9 m/z 1713.15, {(Na⁺)[C₆₀H₄₈N₁₈O₆S₂Ti(IV)Pt(II)₂Cl₂]}⁺; and 10 m/z 1963.15, {[C₆₀H₄₇N₁₈O₆S₂Ti(IV)Au(III)₂Cl₂]}⁺.

UV-Vis spectra were collected of Ti(DefNEtTrp)₂, TiAu₂(DefNEtTrp)₂ and TiPt₂(DefNEtTrp)₂. The metal complexes were prepared in situ by diluting as required, relatively high concentrations of stock solutions of DefNEtTrp, K₂[TiO(C₂O₄)₂], KAuCl₄, and K₂PtCl₄. A 2 mL Ti(DefNEtTrp)₂ solution was prepared by reacting 50 μM of DefNEtTrp with K₂[TiO(C₂O₄)₂](50 μM) in 95% DMF and the solution was left to react overnight. TiAu₂(DefNEtTrp)₂ and TiPt₂(DefNEtTrp)₂ solutions (2 mL) were prepared by reacting 50 μM of Ti(DefNEtTrp)₂ (prepared as indicated above) with KAuCl₄ or K₂PtCl₄ (100 μM) in 95% DMF and left to react overnight.

National Cancer Institute Cancer Cell Line (NCI 60) Screen of DefNEtTrp. Details of the methodology for NCI 60 cell line screening are described at http://dtp.nci.nih.gov/branches/btb/ivclsp.html. The cells, grown in supplemented RPMI-1640 medium, are seeded in 96 well plates at an appropriate density and incubated for 24 h. The test compounds are dissolved in DMSO and incubated with cells at a single dose of 10.0 μM for 48 h. The assay is terminated by addition of cold trichloroacetic acid, and the cells are fixed and stained with sulforhodamine B. Bound stain is solubilized, and the absorbance is read on an automated plate reader. Compounds which exhibit significant growth inhibition in the one dose screen are evaluated against a 57 cell panel at five concentration levels (0.01, 0.1, 1, 10 and 100 μM). Using the seven absorbance measurements [time zero, (Ti), control growth, (C), and test growth in the presence of drug at the five concentration levels (Tf)], the percentage growth was calculated at each of the drug concentrations levels as: [(Tf−Ti)/(C−Ti)]×100 for concentrations for which Tf≥Ti and [(Tf−Ti)/Ti]×100 for concentrations for which Tf<Ti. Three-dose response parameters (GI₅₀, TGI, and LC₅₀) were calculated for each of the experimental agents in five dose assay. Growth inhibition of 50% (GI₅₀) was calculated from 100×[(Tf−Ti)/(C−Ti)]=50, which was the drug concentration resulting in a 50% reduction in the net protein increase (as measured by sulforhodamine B, SRB staining) in control cells during the drug incubation. The total growth inhibition (TGI) was calculated from Tf=Ti, which was the drug concentration resulting in total growth inhibition and signified the cytostatic effect. The 50% cellular death (LC₅₀) was calculated from 100×[(Tf−Ti)/Ti]=−50, indicating a net loss of cells following treatment which indicated the concentration of drug resulting in a 50% reduction in the measured protein at the end of the drug treatment as compared to that at the beginning. Values were calculated for each of these three parameters at the level of activity; however, if the effect did not reach to the level of activity, the value of parameter was expressed as less than the minimum concentration tested, or if the effect exceeded the level of activity, the value of parameter was expressed as greater than the maximum concentration tested. Log GI₅₀, log TGI, and log LC₅₀ are the logarithm molar concentrations producing 50% growth inhibition (GI₅₀), a total growth inhibition (TGI), and a 50% cellular death (LC₅₀), respectively.

Jurkat cell culturing and cell viability studies. Jurkat cells are nonadherent cells, and they were cultured in an RPMI-1640 media (supplemented with 10% FBS (v/v) and 1% antibiotic solution (v/v) of penicillin/streptomycin) in non-tissue-culture treated 25 mL flasks according to the known protocol provided by the supplier ATCC. The cell passaging was done at 70% confluency by taking 3 mL of cells and diluting to 25 mL of fresh media. The cells were incubated at 37° C. in 5% CO₂ atmosphere.

Jurkat cytotoxicity of DefNEtTrp (6), Def (5), NEtTrp (4), Trp, and 1:1 combinations of 5 with 4 and 5 with Trp was determined by the colorimetric 3-(4,5-dimethylthiazol-2-yl)-2,5-diphenyltetrazolium bromide (MTT) assay. Incubation conditions were the same in all the steps. Cells in 80-95% confluence (grown in standard circular tissue culture dishes, BD Falcon) were separately seeded into 96-well plates using phenol red free RPMI-1640 media (containing 10% FBS and 1% streptomycin/penicillin) in a volume of 50 μL at a concentration of 5.0×10⁵ cells/mL. Following this procedure, the Jurkat cells were treated immediately. Stock solution of the compounds were first prepared in DMF at 5 mM and stored at 4° C. for up to 30 days. The stock solution was diluted to specific concentrations using 1×PBS (0.2-20 μM) while DMF was maintained at 1% (v/v). 50 μL of diluted solutions were added in all cell containing wells with at least six replicates per concentration (n=6). The final percentage (v/v) of DMF was 0.5%. The cells were also co-treated with 5 with 4 and 5 with Trp in a 1:1 mole equivalent ratio over the same concentration range but as a combined concentration of both compounds. Control wells in the plates consisted of one lane of cells treated with media alone including 0.5% DMF (v/v) as the measure of 100% viable cell growth in the media. Another control lane consisted of no cells with media including 0.5% DMF (v/v) as a measure of the background with no viable cells. The plates were incubated for 72 h. At 4 h before completion of the incubation time, 25 μL of MTT solution (sterile filtered 5 mg/mL solution, dissolved in 1×PBS buffer) was added to each well. During addition of the MTT solution, the plates were protected from light. After completion a 50 μL portion of 4% (w/v) SDS solution (sterile filtered, dissolved in Tris buffer 1M, pH=10) was added to each well and incubated to solubilize formazan crystals after MTT cell labeling. The absorbance of each well was measured at 570 nm (absorbance of the formazan product at pH >10.0) and 800 nm (as a background correction) using a Tecan plate reader. The absorbance of all wells was compared to the absorbance of the untreated cells with the MTT set as the 100% viable cell standard and the absorbance of the no cell control set as the 0% viable cell standard. Nonlinear regression in GraphPad Prism 8 was utilized to fit the growth curve over the various drug concentrations to determine the half-maximal inhibitory concentration (IC₅₀). All cell viability experiments, including non-Jurkat cells, were repeated at least once for biological duplicates. To confirm cell death at the higher concentration dosages of the compounds and combinations, dead cells were blue stained with trypan blue and visually inspected under the microscope.

Fe(III) supplementation cell viability study. Jurkat cells were seeded into 96-well plates, as indicated in the MTT assay described above. One group of cells was supplemented with 2 μM [Fe(citrate)₂]⁵⁻ (prepared in situ) while another was supplemented with 5 μM [Fe(citrate)₂]⁵⁻. Another group of cells were treated with media alone. After 2 h incubation, half of the cells supplemented with [Fe(citrate)₂]⁵⁻ were treated with 2 μM DefNEtTrp while the other half was treated with media alone. The final percentage of DMF was maintained at 0.5% (v/v). The MTT assay was performed with these samples (n=6). A student t-test was performed to evaluate the difference in the viability of cells treated with DefNEtTrp and supplemented with and without [Fe(citrate)₂]⁵⁻.

MRC-5 cell viability studies. A thawed stock of MRC-5 cells was washed with 1×PBS and resuspended in phenol red DMEM media (supplemented with 10% FBS and 1% penicillin-streptomycin) and then seeded in a 100 mm×20 mm (complete, O.D.×H) petri dish and grown in a 5% (v/v) CO₂ humidified atmosphere at 37° C. At least three passages were performed to ensure the integrity of the cells. After this point, cells were collected, thoroughly washed with 1×PBS, and then resuspended in phenol red-free DMEM (supplemented with 10% FBS, 1% penicillin-streptomycin, and 2.4 mM L-glutamine) at a 2.0×10⁵ cells/mL concentration. A volume of 50 μL of cells was seeded into 96-well plates. Cells were incubated for 24 h. Stock solutions of DefNEtTrp were prepared fresh in DMF at concentrations of 20,000, 10,000, 5,000, 2,000, 1000, 200, 80, and 40 μM. The solutions were diluted 100-fold in 1×PBS to obtain a second set of stock solutions with 1% DMF(v/v). The MRC-5 cells were treated with 50 μL of DefNEtTrp compound solution from the second set of stock solutions to obtain final concentrations of 100, 50, 25, 10, 5, 1, 0.4, and 0.2 μM with 0.5% DMF (v/v). Control wells were the same type of controls as in the Jurkat cell work. Cells were incubated for 68 h and the MTT assay was performed exactly as described for the Jurkat cells.

CO-ADD hemolysis assay. Dry compounds ranging from 1-2 mg were delivered to the CO-ADD. The compounds were solubilized to a stock concentration of 10 mg/mL in DMSO. Samples were serially diluted 1:2 fold in water to final testing concentrations of 32, 16, 8, 4, 2, 1, 0.5, 0.25 g/mL while keeping the final DMSO concentration to a maximum of 0.32% (v/v). Each sample concentration was prepared in polypropylene 384-well plates (Corning 3657) for hemolysis assays, all in two duplicate plates (n=2). All sample preparation was conducted using liquid handling robots. Human whole blood (Australian Red Cross) was washed three times with three volumes of 0.9% NaCl (w/v) and resuspended in a concentration of 0.5×10¹ cells per mL, determined by manual cell count in a Neubauer hemocytometer. Washed cells were added to the compound-containing plates for a final volume of 50 μL. After a 10 min shake on a plate shaker the plates were then incubated for 1 h at 37° C. After incubation, the plates were centrifuged at 1000 g for 10 min to pellet cells and debris, 25 μL of the supernatant was then transferred to reading plates (384 well, polystyrene plated (PS), Corning CLS3680), with hemolysis determined by measuring the supernatant absorbance at 405 mm (OD₄₀₅) using a Tecan M1000 Pro monochromator plate reader. HC₁₀ and HC₅₀ (concentration at 10% and 50% hemolysis, respectively) were calculated by curve fitting the inhibition values vs. log(concentration) using a sigmoidal dose-response function with variable fitting values for the top, bottom, and slope. The maximal percentage of hemolysis is reported as Dₘₐₓ. Hemolysis samples are classified by HC₁₀≤32 μg/mL. Melittin (Sigma M2272) was used as a positive hemolytic control on each plate and exhibited HC₁₀ and HC₅₀ values within the expected range. Melittin was used in 8 concentrations in 2-fold serial dilutions with 50 g/mL being the highest concentration.

Ferroptosis mechanistic cell viability study. Jurkat cells were seeded into 96-well plates, as indicated in the MTT assay described above The cells were treated with 10 μM of the ferroptosis inhibitor ferrostatin-1 (Fer-1). Another group of cells were treated with media alone. After 1 h incubation, half of the cells supplemented with Fer-1 were treated with 2 μM DefNEtTrp or 25 μM [Fe(citrate)₂]⁵⁻, as positive control, while the other half was treated with media alone. The final percentage of DMF was maintained at 0.5% (v/v). The MTT assay was performed with these samples (n=6). A student t-test was performed to evaluate the difference in the viability of cells treated with DefNEtTrp or [Fe(citrate)₂]⁵⁻ and supplemented with and without Fer-1.

Apoptosis mechanistic cell viability study. Jurkat cells were seeded into 96-well plates, as indicated in the MTT assay described above. The cells were treated with 10 μM of the apoptosis inhibitor Q-VD-OPh (Q-V-O). Another group of cells were treated with media alone. After 1 h incubation, half of the cells supplemented with Q-V-O were treated with 2 μM DefNEtTrp or 2 μM cisplatin, as positive control, while the other half was treated with media alone. The final percentage of DMF was maintained at 0.5% (v/v). The MTT assay was performed with these samples (n=6). A student t-test was performed to evaluate the difference in the viability of cells treated with DefNEtTrp or cisplatin and supplemented with and without Q-V-O.

### DISCUSSION

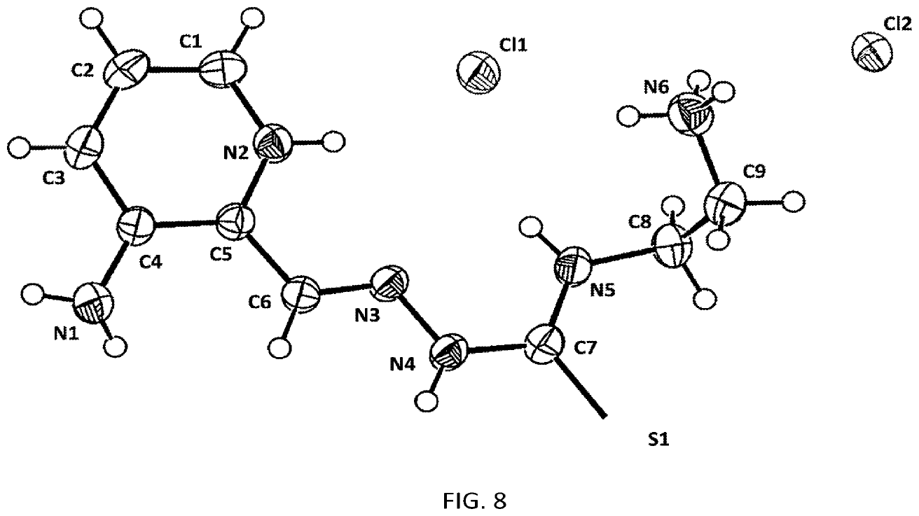

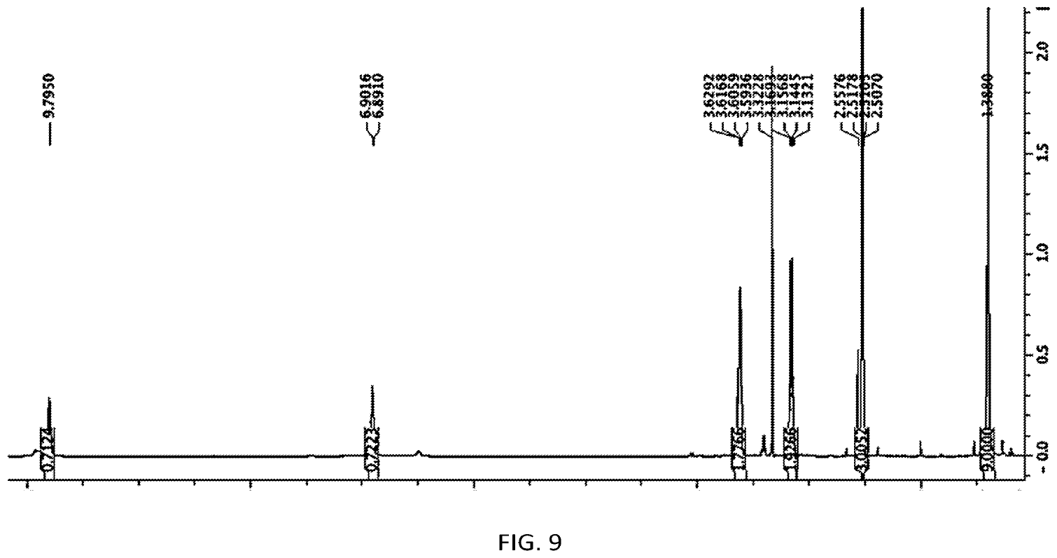

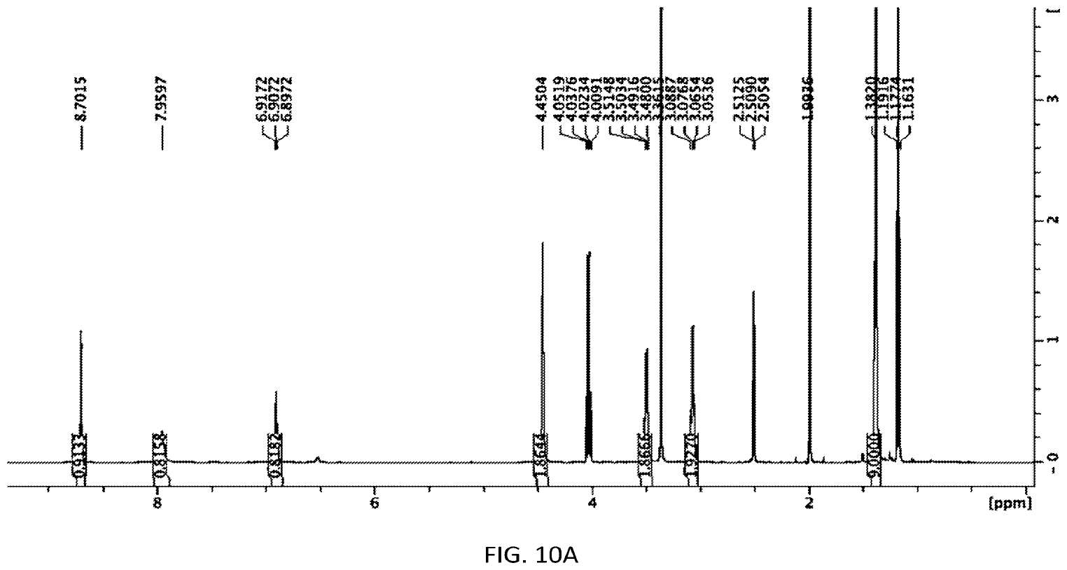

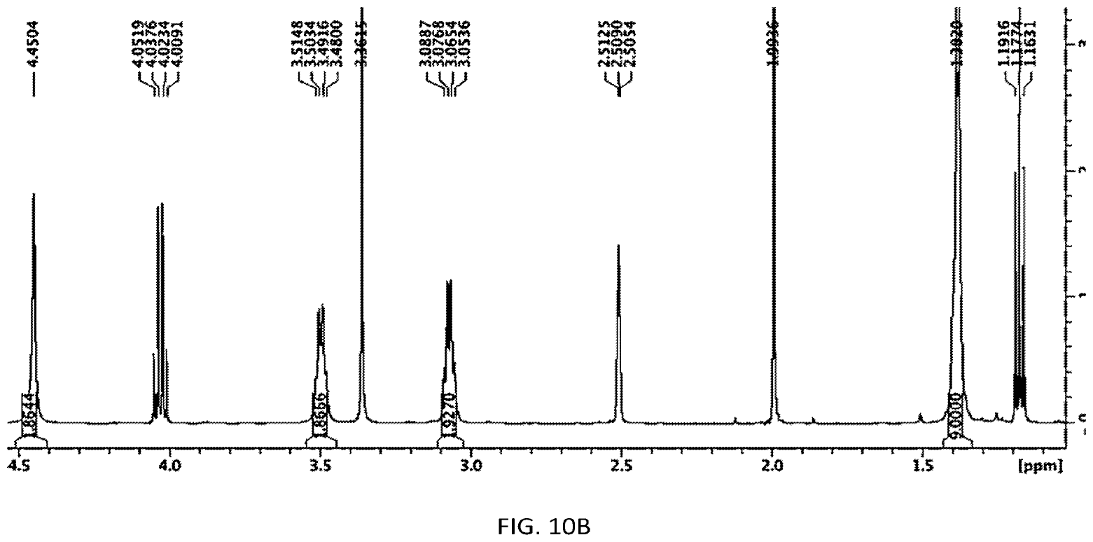

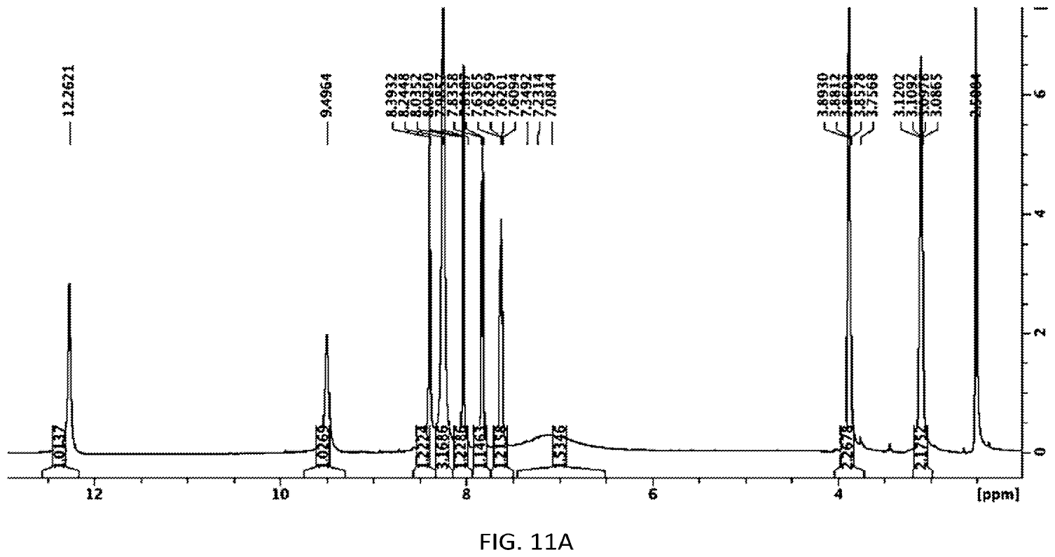

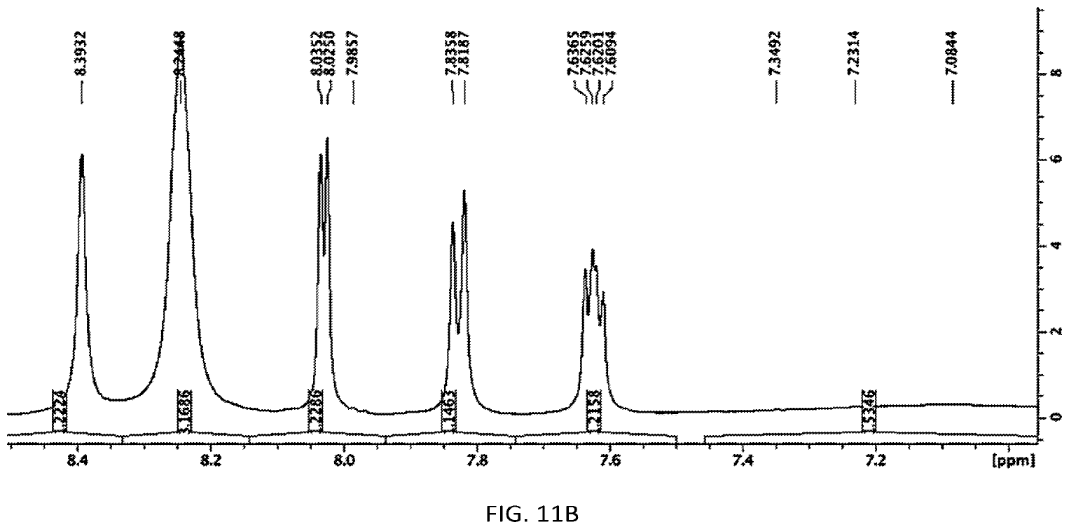

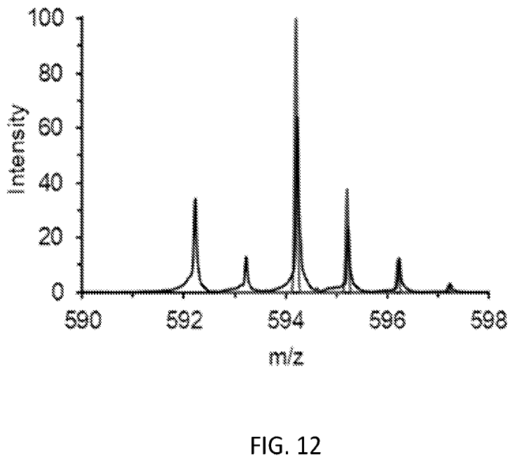

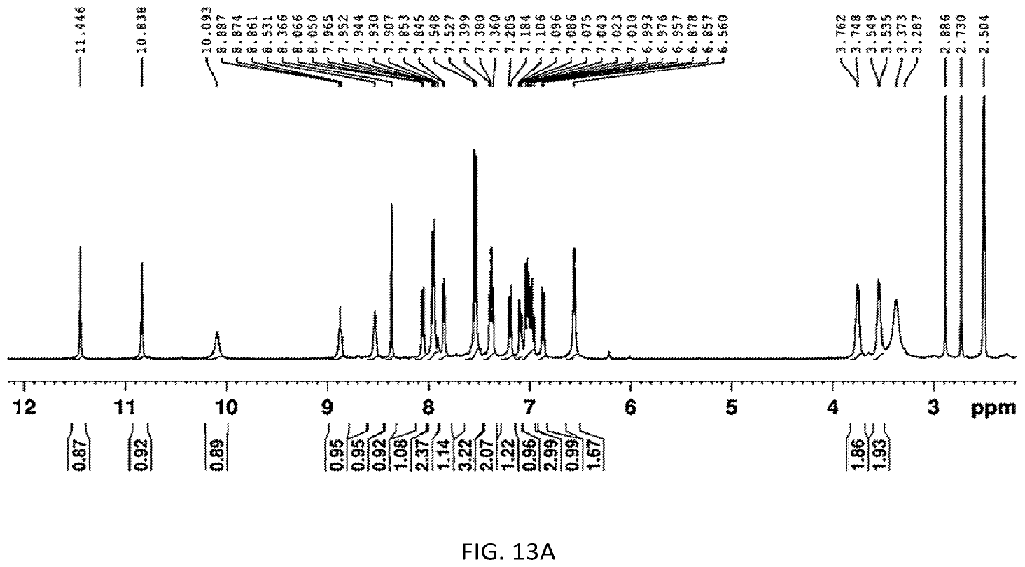

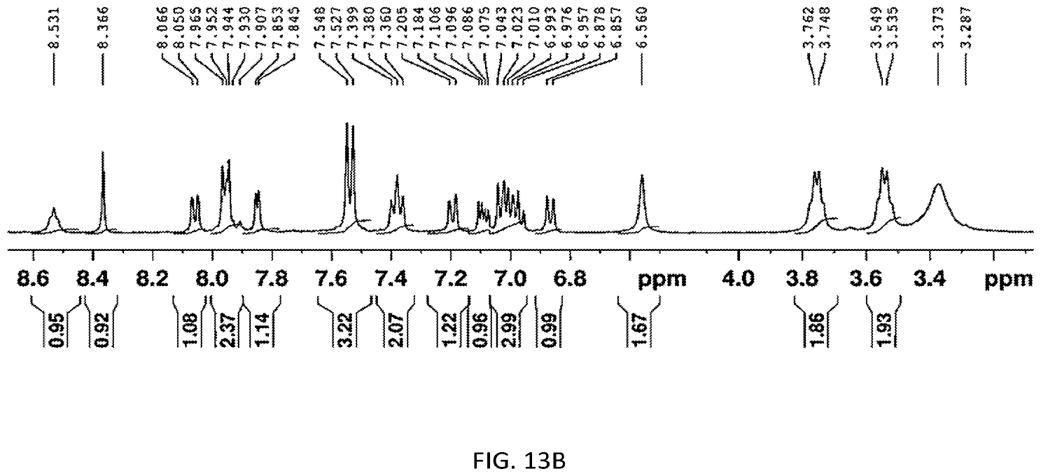

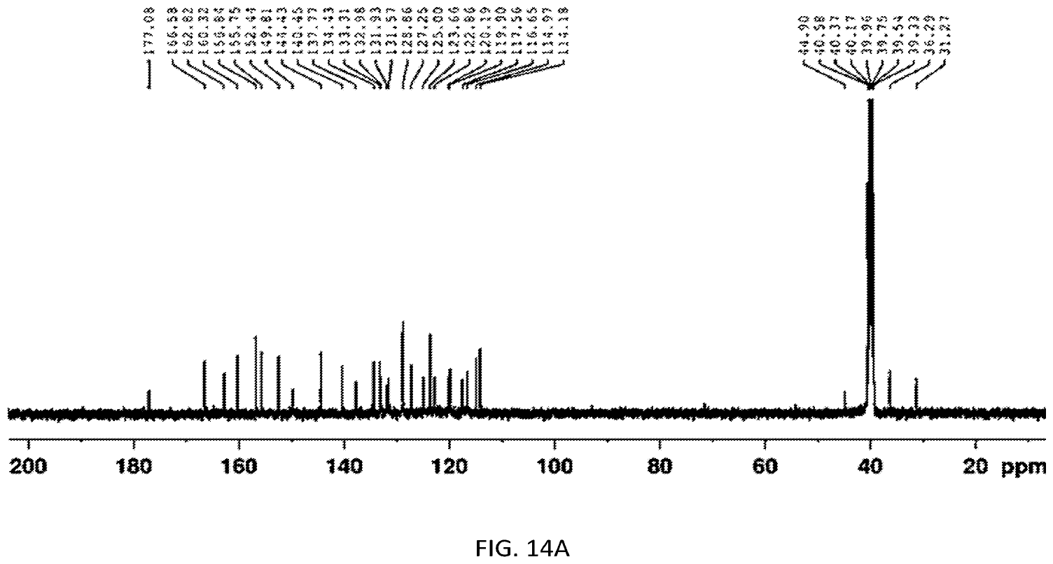

Synthesis of the dual chelator ligand. In this disclosure, a dual chelator ligand was designed coupling the Fe binding Def and Trp moieties to exploit their difference in Lewis basicity for anticancer application. A facile synthetic route was used to conjugate the two chelators with an ethylenediamine linker, resulting in a high yield product. The synthesis of DefNEtTrp (6) was accomplished through a four-step synthetic route (see Scheme 1 below) adapting a literature protocol (see, Kallus, et al., 2019, Synthesis and biological evaluation of biotin-conjugated anticancer thiosemicarbazones and their iron(III) and copper(II) complexes. J. Inorg. Biochem., 190, 85-97). First, nucleophilic addition of N-Boc-ethylenediamine to carbon disulfide followed by methylation with iodomethane was performed to yield methyl-N-(2-tert-butoxycarbonylaminoethyl)-dithiocarbonate (2). Second, nucleophilic addition-elimination reaction with hydrazine monohydrate yielded thiosemicarbazide (3). N-ethyleneamine triapine dihydrochloride (NEtTrp-2HCl) (4) was then obtained through a condensation reaction between 3 and 3-amino-picolinaldehyde in ethanolic HCl. These conditions generated quality X-ray diffracting single crystals of 4 (FIGS. 7 and 24; CCDC Deposition number 2216216). The DefNEtTrp (6) conjugate compound was obtained via the carbodiimide-mediated amide coupling of 4 and Def (5). Each of the intermediates was confirmed by ¹H NMR spectroscopy (spectra are shown FIGS. 8, 9A-9B, and 10A-10B). DefNEtTrp was characterized by MALDI-TOF mass spectrometry (MS), ¹H and ¹³C NMR, and C,H,N elemental analysis. The measured mass distribution of the compound matched the theoretical distribution of {(H⁺)[C₃₀H₂₇N₉O₃S]}¹⁺, (m/z=594.23) (FIG. 11). Additionally, ¹H and ¹³C NMR data (see FIGS. 12A-12B and 13A-13B) and C,H,N elemental analysis confirmed the successful synthesis of the compound in high purity.

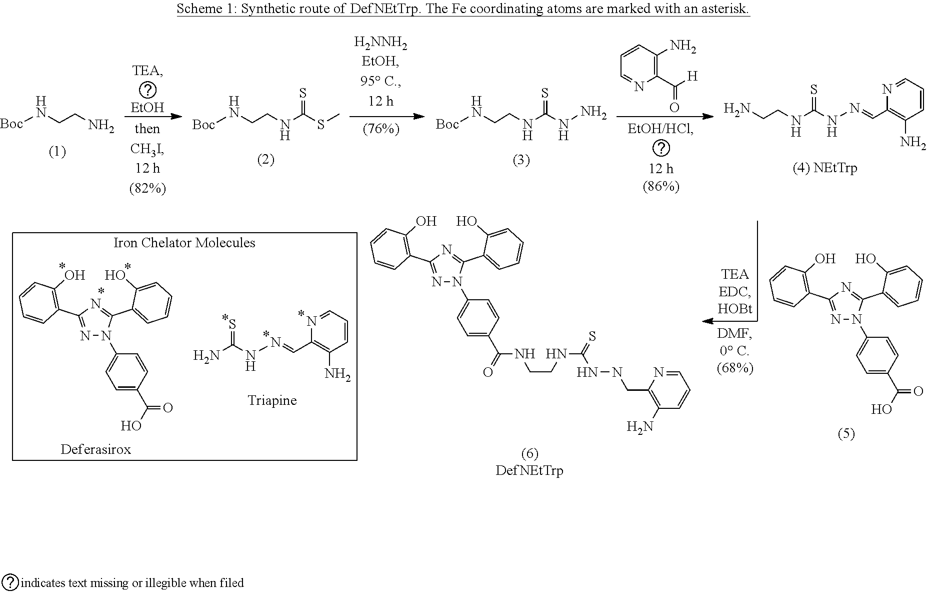

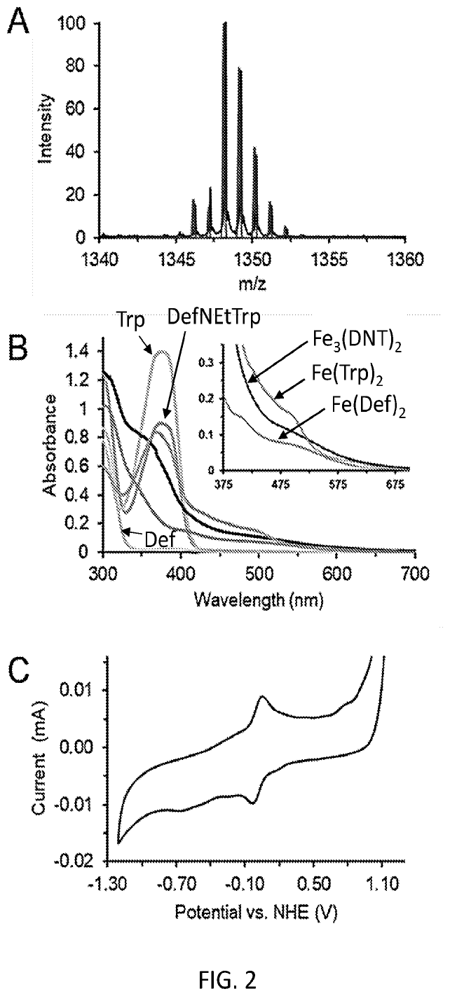

Characterization of the in situ Fe(III) complexation by DefNEtTrp. Fe(III) complexation by DelNEtTrp was explored using a labile Fe(III) source. Due to the limited water solubility of the ligand, its in situ Fe(III) complexation was performed in DMF and diluted in aqueous solution as needed for different characterizations. The initial complexation reactions and subsequent dilutions were left to equilibrate for ≥24 h. A 100 μM solution (moderately soluble), based on ligand concentration, was prepared by dilution to a 2.1% DMF (v/v) final and pH adjusted to pH 7.4. Positive ion MALDI-TOF MS (see FIG. 2A) revealed a peak with the highest m/z value at 1348.10 for the species {C₆₀H₄₈N₁₈O₆S₂Fe₃}⁺, indicative of a putative Fe₃(DefNEtTrp)₂ complex (7). The theoretical spectrum overlayed very well with the experimental spectrum and exhibited the expected isotopic distribution for the presence of 3 Fe ions.

Comparative analysis of UV-Vis spectra (see FIG. 2B) for Def, Trp, DefNetTrp, Fe(Def)₂, Fe(Trp)₂, and Fe₃(DefNetTrp)₂ was performed to evaluate Fe(III) coordination by DefNEtTrp. The spectra were collected of solutions at a set ligand concentration of 50 μM (95%:5% (v/v) DMF:Water solutions to avoid solubility issues). The metal complexes were prepared in situ with excess Fe(III) to try to ensure complete metal complexation. The spectra for the metal complexes were corrected (with virtually little change) by subtracting concentration normalized spectra for non-complexed Fe(III) in the same solvent system. The Fe(Def)₂ species exhibits ligand to metal charge transfer (LMCT) absorbance shoulders at 406 nm (ε=5,940 M⁻¹ cm⁻¹) and 501 nm (ε=2,800 M⁻¹cm⁻¹). The Fe(Trp)₂ species revealed a quenching of the n 43 π* broad transitions of the pyridine ring and the thiosemicarbazide group of Trp in the 325 to 433 nm wavelength range. There were also LMCT absorbance shoulders at 497 nm (ε=5,980 M⁻¹ cm⁻¹) and 601 nm (ε=970 M⁻¹ cm⁻¹). The Fe₃(DefNetTrp)₂ species revealed a quenching of the n→π* broad transitions characteristic of the Trp moiety in the 330 to 433 nm wavelength range. There were LMCT absorbance shoulders at 492 nm (ε=4,440 M⁻¹cm⁻¹) and 601 nm (ε=1,200 M⁻¹cm⁻¹), characteristic of Fe(III) coordination by the Def and Trp moieties.

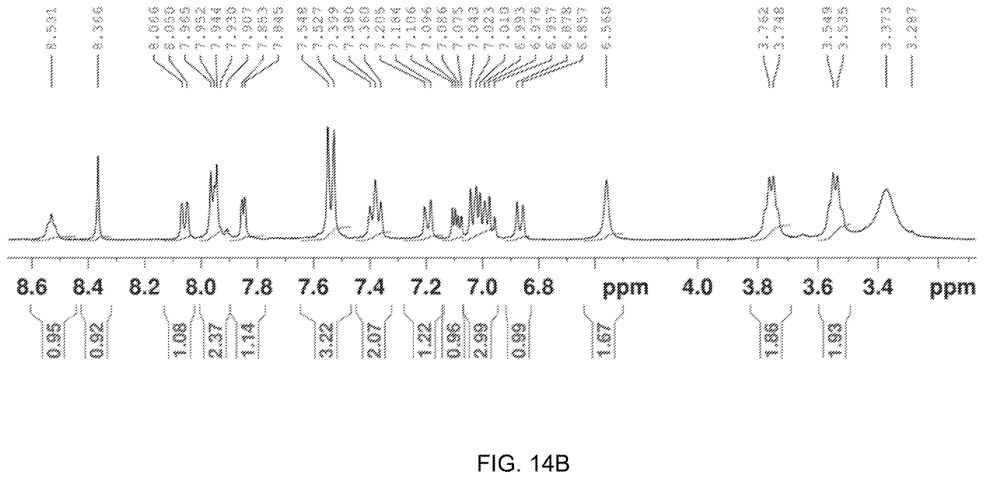

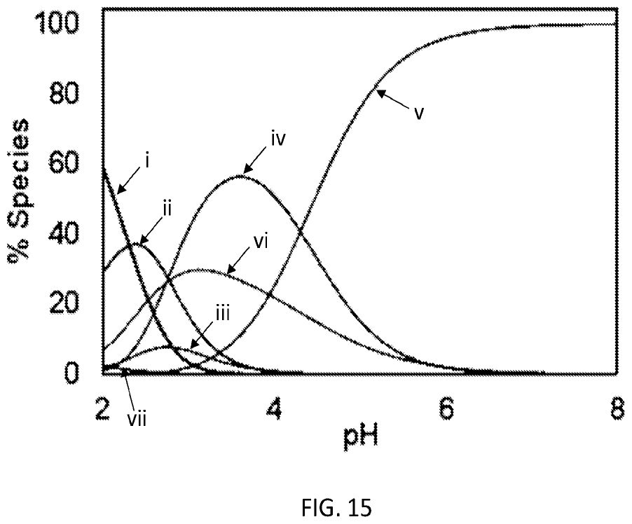

Although the exact structure of Fe₃(DefNEtTrp)₂ has not been determined (and is unnecessary for the purposes set forth herein), a reasonable assumption can be deduced based on ligand affinity and speciation studies. At pH 7.4, Def and Trp displayed a high affinity for Fe(III) but the affinity of Def was far higher (log β for Fe(TIT)(Trp)₂=26.3 vs log 3 for Fe(III)(Def)₂=36.9) (Enyedy et al., 2004, Interaction of triapine and related thiosemicarbazones with iron(III)/(II) and gallium(III): A comparative solution equilibrium study. Dalton Trans. 40 (22): 5895-905; Steinhauser et al., 2004, Complex formation of ICL670 and related ligands with Fe(III) and Fe(II). Eur. J. Inorg. Chem. 2004 (21): 4177-4192). In interaction with DefNEtTrp, Fe(III) is expected to preferentially bind first to the Def moiety and then bind to the Trp moiety. Using a pH-dependent speciation model for 50 μM Fe(III) in interaction with Def and Trp (both at 100 μM), the results of which are shown in FIG. 14), this model revealed that at pH 7.4, Fe(III) exclusively bound Def as the Fe(Def)₂²⁻ species, which was also the case for micromolar levels of Fe(III) in general in which the metal:Def:Trp ratio was maintained 1:2:2. For the Fe₃(DefNEtTrp)₂ molecular formula observed by MALDI-TOF MS, a proposed general structure for Fe(III) complexation is shown in Scheme 2 and more detailed structure is shown in FIG. 15. In this structure Fe(III) is coordinated to Def in a 1:2 Fe:Def modality presumably with meridional ligand arrangement as observed for Fe(Def)₂ and Ti(Def)₂. The additional two Fe(III) ions in this structure would be bound to the two Trp moieties in each DefNetTrp in a square planar arrangement. The Trp moiety provides tridentate coordinating with the fourth coordinating atom being either a solvent molecule (if water, then possibly metal-hydrolyzed to hydroxide) or chloride from the original FeCl₃ reactant (Saxena et al., 2018, Exploring titanium(IV) chemical proximity to iron(III) to elucidate a function for Ti(IV) in the human body. Coord. Chem. Rev. 363: 109-125). Significantly, in the MALDI-TOF spectrum the Fe(DefNEtTrp)₂ {(H⁺)₂[C₆₀H₅₀N₁₈O₆S₂Fe]}⁺ and Fe₂(DefNEtTrp)₂ {C₆₀H₄₉N₁₈O₆S₂Fe₂}⁺ species were observed (see FIG. 15), which could have arisen from ionization lability of the Fe ions at the Trp moieties within the instrument's source (the results of these assays having m/z=1240.41, {(H⁺)₂[C₆₀H₅₀N₁₈O₆S₂Fe]}⁺ (Fe(DefNetTrp)₂) and m/z 1293.32, {C₆₀H₄₉N₁₈O₆S₂Fe₂}⁺ ((Fe₂(DefNetTrp)₂). There was no basis for believing that any species with Fe(III)-free Trp moiety existed in solution under the synthetic conditions employed. A monoTrp coordination of Fe(III) was typically only observed in acidic conditions. A higher order structure in which an Fe(III)-free Trp moiety from DefNEtTrp or from Fe(DefNEtTrp)₂ coordinates to the “square planar” Fe(III) ions from a Fe₃(DefNEtTrp)₂ unit to form a meridional Trp arrangement was not observed due to steric hindrance posed by the Def moiety. Another (more likely) possibility was that under the reaction conditions of stoichiometric or excess Fe(III) used, uncomplexed labile Fe(III) rapidly saturated the Trp moieties.

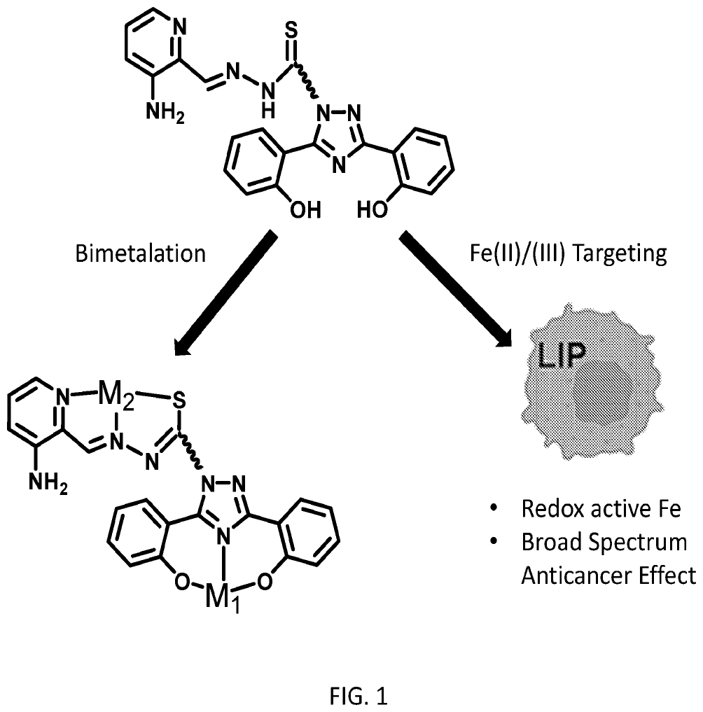

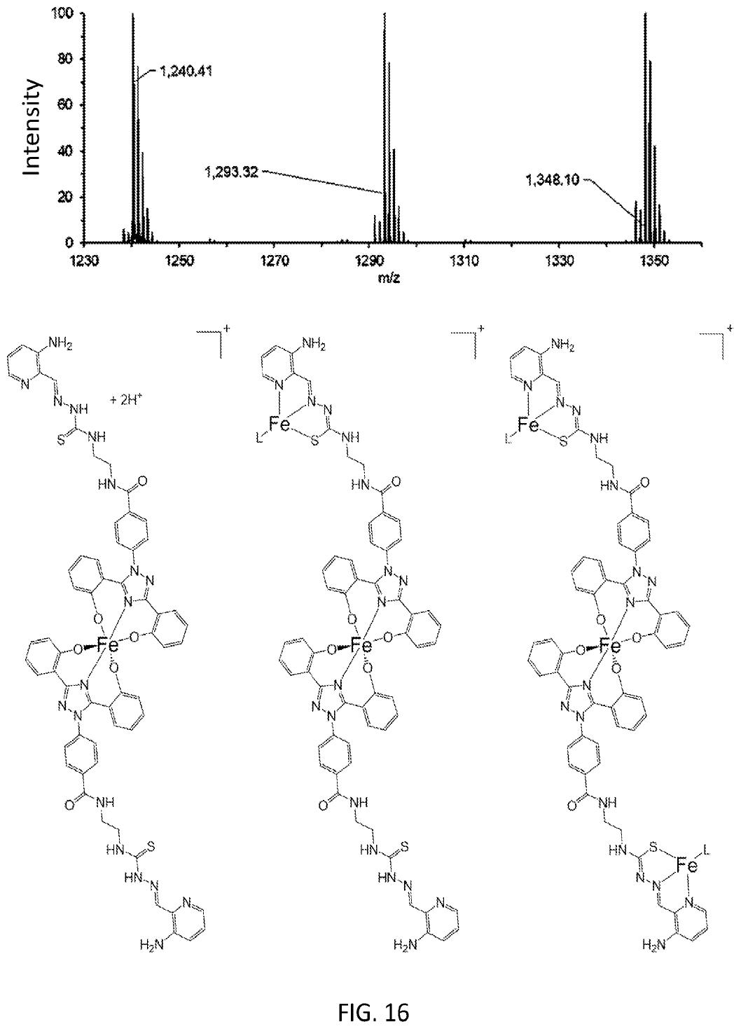

The Fe(III) redox behavior in Fe₃(DefNEtTrp)₂ provided structural and activity insight. A cyclic voltammetric analysis of 6 mM Fe₃(DefNEtTrp)₂ (see FIG. 1) was recorded in DMF solution with added hydroxide to approximate pH 7.4 and compared with that of Fe(Def)₂ as assessed herein and Fe(Trp)₂ collected under similar conditions (Popovic-Bijelic et al., 2011, Ribonucleotide reductase inhibition by metal complexes of triapine (3-aminopyridine-2-carboxaldehyde thiosemicarbazone): a combined experimental and theoretical study. J. Inorg. Biochem. 105 (11): 1422-31). Fe(Def)₂ was seen to undergo an irreversible reduction to the Fe(II) form at Eₚₐ=−0.60 V vs Normal Hydrogen Electrode (NHE) (see FIG. 16). In the Fe(Def)₂ complex, Fe(III) was bound with high affinity and was very solution stable, particularly in aqueous solution at pH 7.4. However, reduction to the Fe(II) form appeared to be quite unstable. Previous aqueous speciation studies have shown that Fe(II) partially dissociates from Def at pH 7.4 but was more stable at basic pH. Fe(Trp)₂ has a reversible redox couple of E_{1/2}=+0.01 V vs NHE. As an intermediate/soft Lewis base Trp has a high affinity for Fe(II) (log β for Fe(II)(Trp)₂=22.55) at pH 7.4 whereas the hard Lewis base Def has a significantly lower affinity (estimated log β for Fe(II)(Def)₂=14.0). Fe₃(DefNEtTrp)₂ has an irreversible reduction at Eₚₐ=−0.56 V vs NHE, characteristic of Fe(III) coordination to the Def moiety. It also has a reversible redox couple of E_{1/2}=+0.103 V vs NHE with a peak potential separation of ΔEₚ=30 mV, indicative of a two-electron process. This redox couple was attributed to the two Fe(III) ions coordinated Trp moieties, which explained the two electron process and suggests that Fe coordination in the +2 and +3 oxidation states by the Trp moiety remains intact in these solution conditions.

Scheme 2: Synthetic route for bimetalation of the DefNEtTrp dual chelator (drawn in a simplified manner) and the proposed compound structure.

When preparing heterometallation, the hard Lewis metal was bound first to the dual chelator followed by the soft Lewis metal.

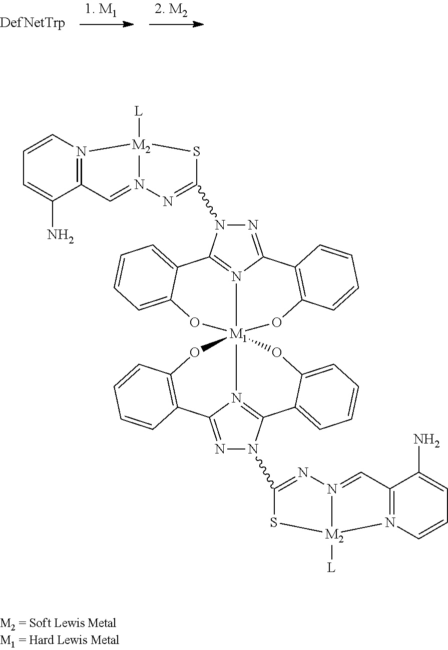

An earlier study exploring Fe(II/III) complexation by Trp and Trp analogues revealed that complexes that exhibited a reversible redox couple less than E_{1/2}=0.2 V vs NHE and not greater than E_{1/2}=0.5 V vs NHE could effectively redox cycle in the cellular environment and reduce the tyrosyl radical of RNR R2 (Plamthottam et al., 2019, Activity and electrochemical properties: iron complexes of the anticancer drug triapine and its analogs. J. Biol. Inorg. Chem. 24 (5): 621-632). Thus Fe₃(DefNEtTrp)₂ was expected to exhibit these properties. This finding was important in the context of the cytotoxic behavior exhibited by DefNEtTrp as discussed below. The ability of DefNEtTrp to coordinate and remove Fe(III) bound to sTf, the main Fe(III) species in serum, was also explored (Williams, & Moreton, 1980, Distribution of iron between the metal-binding sites of transferrin in human-serum. Biochem. J 185 (2): 483-488). Fe(III)-bound sTf plays the vital functions of regulating Fe homeostasis and bioavailability in blood and delivering the metal to all cells in the body. STf features two high affinity Fe(III) binding sites (Li et al., 1996, Rationalization of the strength of metal binding to human serum transferrin. Eur. J. Biochem. 242 (2): 387-393) that undergo a conformational change after Fe(III) binding that limits solvent exposure (Lin et al., 2020, Calorimetric studies of serum transferrin and ovotransferrin-Estimates of domain interactions, and study of the kinetic complexities of ferric ion-binding. Biochemistry 33 (7): 1881-1888; Benjamin-Rivera et al., 2020, Exploring serum transferrin regulation of nonferric metal therapeutic function and toxicity. Inorganics 8 (9): 48), and makes the metal extremely ligand exchange inert (Morgan, 1979, Studies on the mechanism of iron release from transferrin. Biochim. Biophys. Acta—Protein Struct. 580 (2): 312-326). The reaction of a near physiological concentration of Fe₂-sTf (sTf is present in blood at 30 to 60 μM) with equimolar amount of DefNEtTrp with respect to Fe(III) concentration was monitored over 3 days. The characteristic LMCT absorbance (λₘₐₓ 465 nm; ε=5,200 M⁻¹ cm⁻¹ was unchanged, indicating no metal dissociation (Schlabach & Bates, 1975, The synergistic binding of anions and Fe3+ by transferrin. Implications for the interlocking sites hypothesis. J. Biol. Chem. 250 (6): 2182-2188). As further confirmation, the reaction solutions were extensively dialyzed and the concentration normalized UV-Vis spectra showed fully intact Fe₂-sTf. As a potential anticancer therapeutic, this is a significant finding as the ligand would not be able to cause systemic toxicity by scavenging Fe from sTf and disrupting its normal biodistribution.

Characterization of the in situ heterobimetal complexation by DefNEtTrp. DefNEtTrp was also characterized for its utility in the selective synthesis of heterometal compounds with a hard Lewis metal ion at the Def moiety and a soft Lewis metal ion at the Trp moiety. Ti(IV) was selected for this case as the hard metal ion to be combined with the soft d8 metal ions platinum(II) (Pt(II)) and gold(III) (Au(III)) (Housecroft & Sharpe, 2012, Inorganic Chemistry. 4th ed.; Pearson). All three of these metals have cytotoxic properties owing to their distinct chemistry, which could prove valuable for future compound design (Dabrowiak, Metals in Medicine. 2nd ed.; Wiley: 2017; p 91-146).

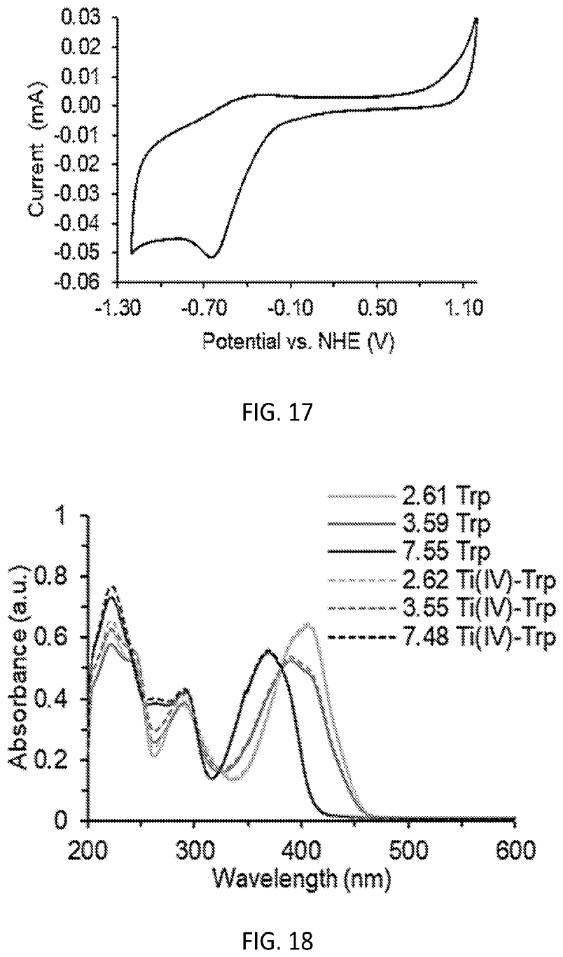

To commence this work, the Trp coordination of Ti(IV) was explored. Trp is part of a group of thiosemicarbazones, a group of ligands that can adopt various binding modes with transition metal ions (Enyedy et al., 2010, Comparative solution equilibrium study of the interactions of copper(II), iron(II) and zinc(II) with triapine (3-aminopyridine-2-carbaldehyde thiosemicarbazone) and related ligands. Eur. J. Inorg. Chem. 2010 (11): 1717-1728). They can act as mono-, bi-, or tridentate ligands although the tridentate modality is typically observed in aqueous solution. As already established, Trp can effectively bind Fe(III), a hard Lewis metal, but is a very poor chelator for transition metals of even higher oxidation states. While V(IV) Trp complexes have been synthesized, Trp has virtually no affinity for V(V) (Kowol et al., 2015, Vanadium(IV/V) complexes of triapine and related thiosemicarbazones: Synthesis, solution equilibrium and bioactivity. J. Inorg. Biochem. 152: 62-73). It is worthwhile noting that stable V(IV) Def dimeric complexes have been prepared and studied (Maurya et al., 2016, Synthesis, characterization, reactivity, catalytic activity, and antiamoebic activity of vanadium(V) complexes of ICL670 (deferasirox) and a related ligand. Eur. J Inorg. Chem. 2016 (9): 1430-1441). Ti(IV) complexation by Trp at micromolar concentrations that maintained a 1:2 metal:ligand ratio was studied by MALDI-TOF MS and combined pH-potentiometric and UV-Vis spectroscopy. No Ti(IV) Trp species were observed by MALDI-TOF; pH-dependent UV-VIS results for the Ti(IV)-Trp system are shown in FIG. 17. These resulted definitively demonstrated that Trp had no significant affinity for Ti(IV) given that the only changes of the spectral features of Trp observed were due to deprotonation upon pH increase.

Synthesis of heterometal DefNEtTrp compounds was achieved by sequential metal ion addition as indicated in Scheme 2. First, excess Ti(IV) was reacted with DefNEtTrp to produce Ti(DefNEtTrp)₂ (8). Due to low aqueous solubility, Ti(DefNEtTrp)₂ could be extracted as a yellow solid via water precipitation. A 100 μM solution was prepared by diluting a 20 mM DMF stock in water to a 5% (v/v) DMF final and it was examined by MALDI-TOF MS. A single Ti-containing peak was observed at 1253.35 m/z for the proposed species {(Na+)[C₆₀H₅₀N₁₈O₆S₂Ti}⁺, in line with the expectation of the Ti(IV) coordinated by a meridional arrangement of the two Def moieties (see FIGS. 2A and 18A) as observed in Ti(Def)₂. The Ti(DefNEtTrp)₂ solid was separately reacted with excess Pt(II) and Au(III) (Gaur et al., 2018, Iron and copper intracellular chelation as an anticancer drug strategy. Inorganics 6 (4): 126). These products could be extracted via water precipitation. The orange-yellow Ti(DefNEtTrp-Pt(II)-Cl)₂ (9) and orange-yellow Ti(DefNEtTrp-Au(III)-Cl)₂ (10) solids were also analyzed by MALDI-TOF MS at 100 μM (5% DMF (v/v)) (see FIGS. 2B-2C). Ti(DefNEtTrp-Pt(II)-Cl)₂ was detected as the species {(Na⁺)[C₆₀H₄₈N₁₈O₆S₂Ti(IV)Pt(T)₂Cl₂]}⁺ (171 3.15 m/z). Ti(DefNEtTrp-Au(TIT)-Cl)₂ was detected as the species {[C₆₀H₄₇N₁₈O₆S₂Ti(IV)Au(III)₂Cl₂]}⁺ (1691.15 m/z).

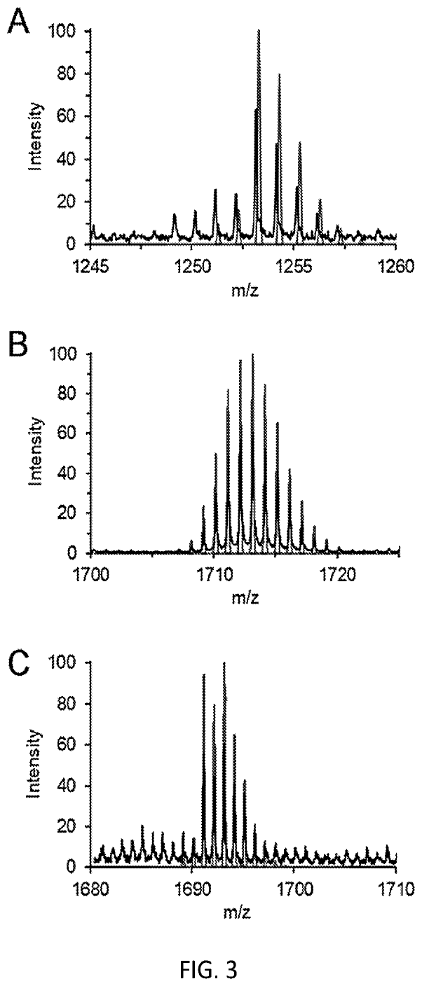

UV-Vis spectra (see FIGS. 3A-3B) were collected for the putative species Ti(DefNEtTrp)₂, Ti(DefNFtTrp-Pt(TT)-Cl)₂, and Ti(DefNFtTrp-Au(III)-Cl)₂ and for Ti(Def)₂. All solutions were prepared at a ligand concentration of 50 μM and the metal complexes were prepared in situ with sequential additional of excess Ti(IV) (left to equilibrate for one day) and then excess of the other metal ions (left to equilibrate for one day) to try to ensure complete metal complexation. Spectra for the metal complexes were corrected (with virtually little change) by subtracting concentration normalized spectra for non-complexed Ti(IV), Pt(II), and Au(III) in the same solvent system. The Ti(Def)₂ species exhibited a LMCT absorbance at 368 nm (ε=13,330 M⁻¹ cm⁻¹). The Ti(DefNEtTrp)₂ species displayed an increase in absorbance in the region corresponding to the n→π* broad transitions of the Trp moieties between the 310 and 380 nm wavelength range. Given that these moieties do not coordinate Ti(IV), the absorbance increase was attributed to growth in LMCT absorbance from the coordination of the Def moieties to Ti(IV). To assess this spectral change, a difference spectrum was produced (see FIG. 3B) by subtracting the concentration-normalized spectrum for metal-free DefNEtTrp. The difference spectrum revealed the LMCT absorbance blue-shifted to 333 nm (ε=14,950 M⁻¹cm⁻¹). The UV-Vis spectra Ti(DefNEtTrp-Pt(II)-Cl)₂ and Ti(DefNEtTrp-Au(III)-Cl)₂ (see FIG. 3A) showed comparable features, a quenching of the n→π* broad transitions characteristic of the Trp moiety in the 330 to 433 nm wavelength range and a growth of absorbances in the 440 to 600 nm wavelength range. To better assess these spectra despite overlapping absorbances, difference spectra (see FIG. 3C) were generated in the exact same way as for Ti(DefNEtTrp)₂. In both difference spectra, the Ti(IV)-centered LMCT absorbance was virtually unchanged in energy, present at 328 nm (ε=14,338 M⁻¹cm⁻¹) in Ti(DefNEtTrp-Pt(II)-Cl)₂ and at 328 nm (ε=18,100 M⁻¹cm⁻¹) in Ti(DefNEtTrp-Au(III)-Cl)₂. A Pt(II)-centered charge transfer absorbance was observed at 448 nm (ε=6,260 M⁻¹cm⁻¹) and a Au(III)-centered charge transfer absorbance was observed at 453 nm (ε=8,780 M⁻¹cm⁻¹). It is unclear if these are LMCT or MLCT absorbances and would require more extensive characterization (Franchini, et al., 1985, Coordinating ability of methylpiperidine dithiocarbamates towards platinum group metals. Polyhedron 4 (9): 1553-1558; Han et al., 2007, Switching between ligand-to-ligand charge-transfer, intraligand charge-transfer, and metal-to-ligand charge-transfer excited states in platinum(II) terpyridyl acetylide complexes induced by pH change and metal ions. Chem. Eur. J. 13 (4): 1231-1239; Lessa et al., 2012, Spectroscopic and electrochemical characterization of gold(I) and gold(III) complexes with glyoxaldehyde bis(thiosemicarbazones): cytotoxicity against human tumor cell lines and inhibition of thioredoxin reductase activity. Biometals 25 (3): 587-98; Nardon et al., 2015, Gold(III)-pyrrolidinedithiocarbamato derivatives as antineoplastic agents. ChemistryOpen 4 (2): 183-91; Haseloer et al., 2021, Ni, Pd, and Pt complexes of a tetradentate dianionic thiosemicarbazone-based O\hat{ }N\hat{ }N\hat{ }S ligand. Dalton Trans. 50 (12): 4311-4322).

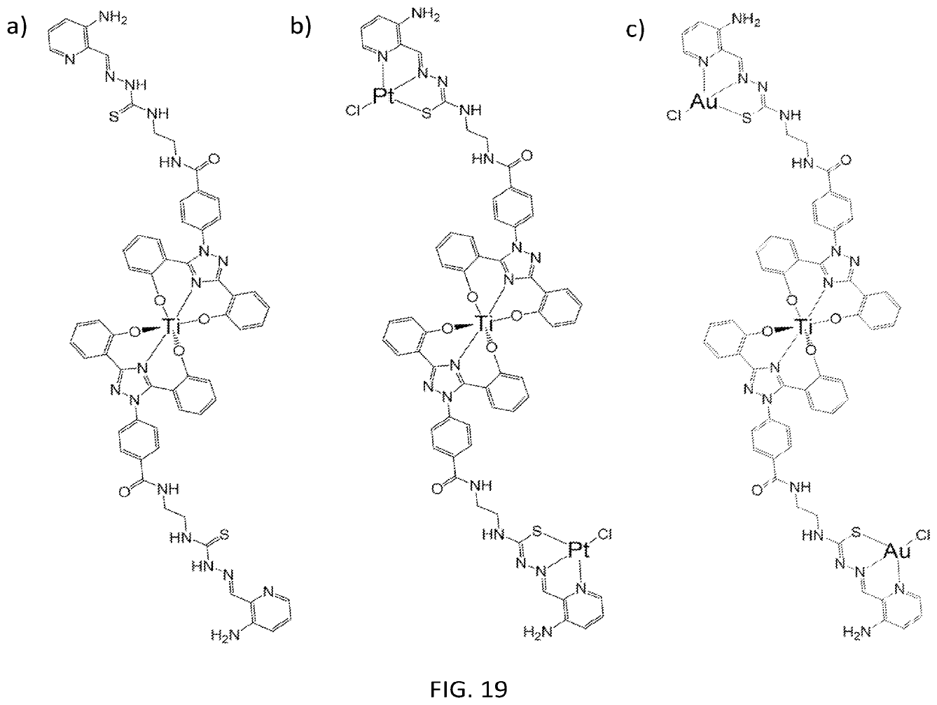

The complexes Ti(DefNEtTrp-Pt(II)-Cl)₂ and Ti(DefNEtTrp-Au(III)-Cl)₂ can be understood to be structurally similar to the Fe₃(DefNEtTrp)₂ complex (see FIGS. 19B-18C), with the hard Lewis Ti(IV) ion bound by meridional arrangement of the Def moieties and the soft metal ions Pt(II) and Au(III) coordinated in a square planar geometry with the Trp moieties as a tridentate chelator and the Cl ligand serving as the fourth coordinating atom. (see FIGS. 18A-18C). Although several square planar Pt(II) compounds have been prepared with Def and Def analogues coordinated in tridentate modality, these compounds were synthesized in organic solvent and no study to date has reported their general aqueous solution stability (Dahm et al., 2015, Tridentate complexes of palladium(II) and platinum(II) bearing bis-aryloxide triazole ligands: A joint experimental and theoretical investigation. Chem. Asian J 10 (11): 2368-2379). A Pt(IV) Def compound was recently synthesized as a prodrug for cellular formation of cisplatin (Pan et al., 2022, Pt(IV)-deferasirox prodrug combats DNA damage repair by regulating RNA N6-methyladenosine methylation. J Med. Chem. 65 (21): 14692-14700). In the complex, Pt(IV) was present in an octahedral geometry with one bound Def ligand. The ligand coordinates Pt(IV) in a monodentate modality via the carboxylate oxygen and not tridentate to the ONO atoms despite Pt(IV) being a harder Lewis acid than Pt(II). This Pt(IV) Def compound was stable for a few hours in PBS buffer before ligand dissociation took place. It would be expected that Pt(II) complexation by Def would be far less stable under the same conditions. No Au Def compounds have been reported.

Overview of the coordination chemistry of DefNEtTrp. Evaluating the metal binding properties of Def and Trp helps to identify how to select the metal ion combination to easily produce exclusive metal binding to each chelator moiety of DefNEtTrp and prevent a mixture of complexes with varying metalation states. Assessment of Fe binding by Def and Trp at pH 7.4 was informative. Fe(III), a hard Lewis acid, had a relatively high affinity to both Def and Trp, with a clear higher preference for Def. In contrast, Fe(II), an intermediate Lewis acid, only had a high affinity to Trp. Calculating the hardness of pertinent metal ions using the Pearson absolute scale (Table 1) provided the following insight using Fe(II) and Fe(III) as benchmarks. Combining metal ions with an absolute hardness equal to or below that of Fe(II) with metal ions with an absolute hardness greater than that of Fe(III) was expected to yield well-defined heterometal DefNEtTrp compounds. Of course, this analysis did not account for the effect of pH-dependent speciation that requires additional assessment particularly of solution stability studies.

TABLE 1
Pearson absolute scale of hardness (η) for metal ions.

| Metal Ion | η |
|---|---|
| Fe(II) | 7.24 |
| Fe(III) | 12.09 |
| Au(I) | 5.64 |
| Au(III) | 6.7 |
| Pt(II) | 5.23 |
| Pt(IV) | 7.25 |
| V(IV) | 9.26 |
| V(V) | 31.44 |
| Ti(IV) | 27.97 |

η = (I − A)/2, where I and A are the ionization potential and the electron affinity, respectively.

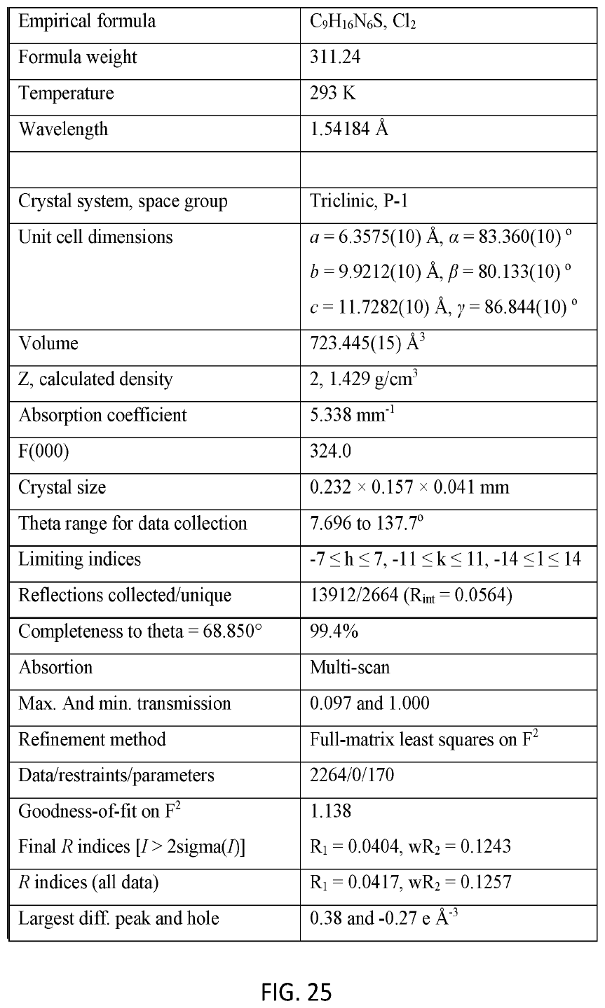

Correlating Fe-binding to the cytotoxicity of DefNEtTrp. DefNEtTrp was submitted to the National Cancer Institute (NCI) 60 human tumor cell line anticancer drug screen, of which 57 were tested (see FIG. 25). The cell lines represented nine human cancers: leukemia, melanoma, lung, colon, central nervous system, ovarian, renal, prostate, and breast cancers. Due to its very promising broad spectrum antiproliferative activity exhibited against the cell lines in a one-dose test of 10 μM administered for 48 h, DefNEtTrp was advanced to a five-dose assay (0.01, 0.1, 1, 10 and 100 μM) for 48 h. Three-dose response parameters were measured: 50% growth inhibition (GI₅₀), total growth inhibition (TGI), and 50% cellular death (LC₅₀). GI₅₀ measured the concentration of a compound that induces 50% inhibition of the cells to reach maximal cell count relative to the initial cell total in no compound treatment conditions. TGI measured compound concentration that retained the cells at the initial cell count used in the screen. LC₅₀ measured the concentration that reduced (kills) 50% of the initial cell total. Table 2 tabulates the calculated averages of GI₅₀ and TGI (including the LC₅₀ values in FIG. 25 and the dose response curves of the nine different panels of cell lines in FIGS. 19A-19I). DefNEtTrp was able to inhibit 50% cell growth (proliferation) of all cell lines at an average GI₅₀ concentration of 1.2 μM. It showed highest antiproliferative sensitivity to leukemia with a GI₅₀ of 0.29 μM and the least sensitivity to renal cancer with GI₅₀ of 4.97 μM. It induced total cell growth inhibition against 77% of the cell lines tested at an average TGI concentration of 8.5 μM. Of these cells, the lowest average TGI (1.78 μM) was observed for leukemia. DefNEtTrp also exhibited cell death capability. It induced 50% cell death against 14% of the tested cell lines, at an average LC₅₀ of 38.9 μM.

TABLE 2
The calculated average 50% growth inhibition (GI₅₀) and tumor
growth inhibition (TGI) of DefNEtTrp in μM drug concentration.

| Cancer cell | GI₅₀ | TGI |
|---|---|---|
| line type | (μM) | (μM) * |
| Leukemia | 0.29 | 1.78 |
| Non-Small Cell Lung | 0.73 | 6.33 |
| Colon | 0.87 | 12.22 |
| CNS | 0.50 | 12.43 |
| Melanoma | 0.61 | 4.73 |
| Ovarian | 1.12 | 9.82 |
| Renal | 4.97 | 13.71 |
| Prostate | 1.14 | 18.2 |
| Breast | 0.45 | 4.84 |

* Average calculated for cell lines in which TGI was observed.

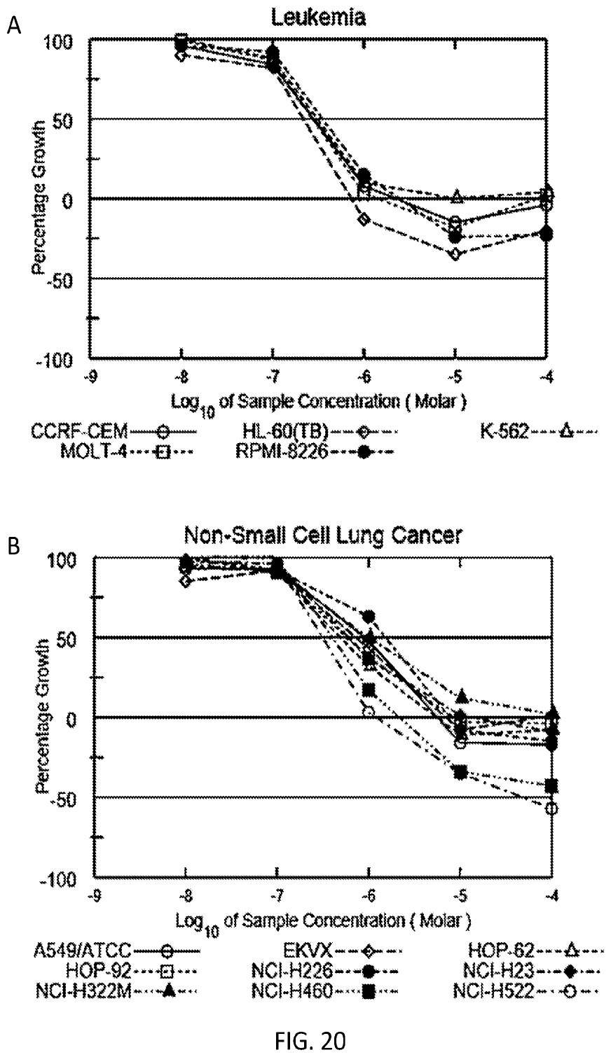

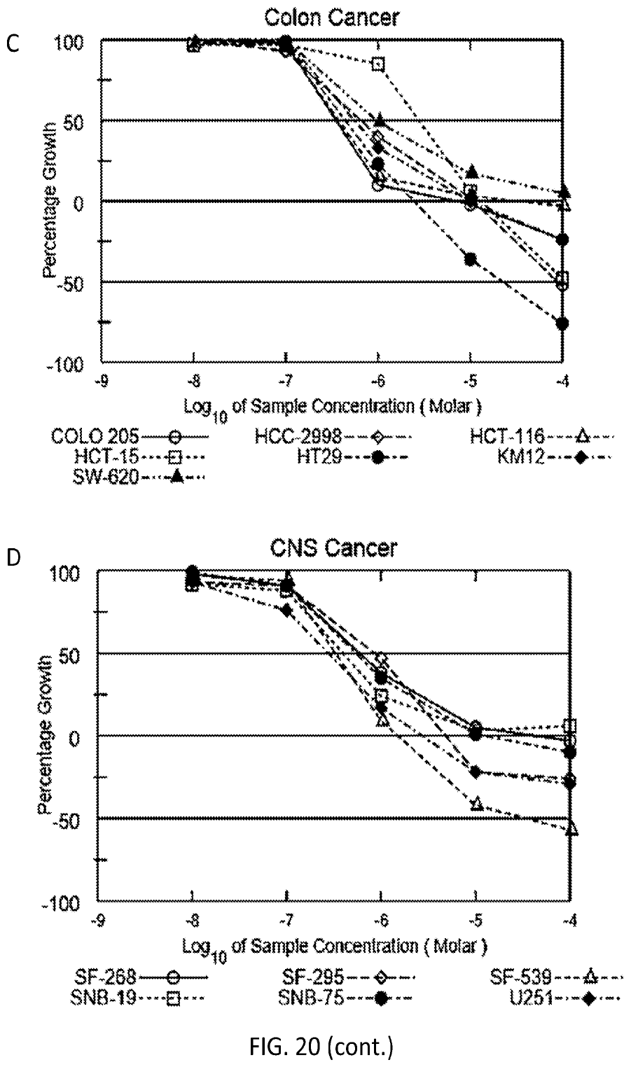

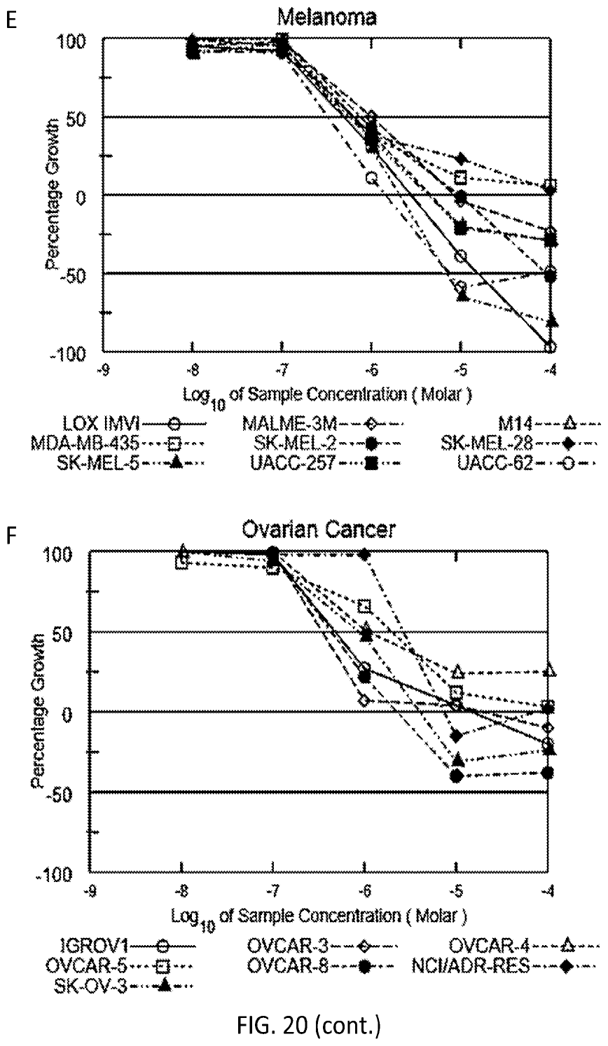

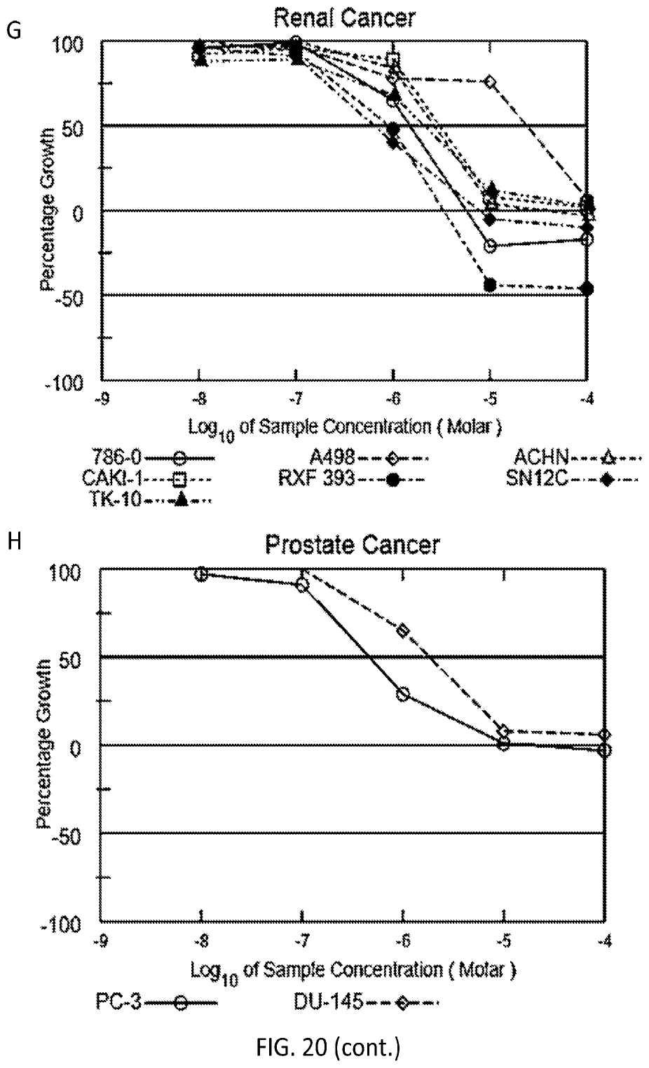

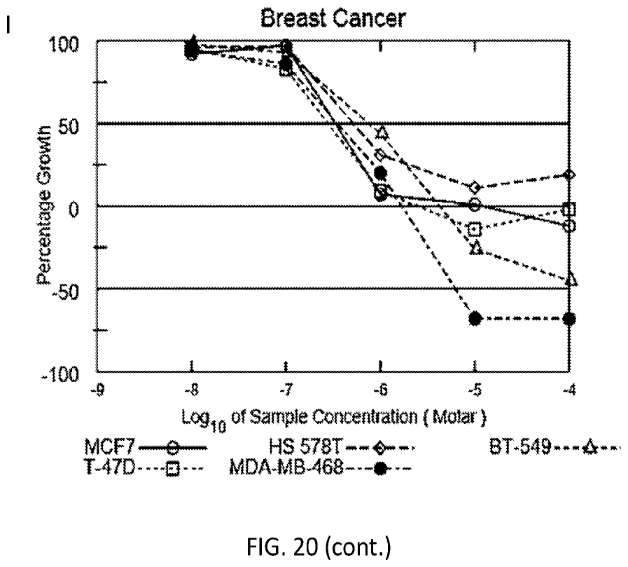

Given the virtually 100% antiproliferative behavior exhibited by DefNEtTrp against all leukemia cell lines in the NCI screen, a more in-depth analysis of its activity was performed against the leukemia Jurkat cell line as a case study. The viability of Jurkat cells was examined in a 72 h treatment with DefNEtTrp, Def, NEtTrp, Trp, 1:1 combination of Def and NEtTrp, and 1:1 combination of Def and Trp. All compounds were examined in the concentration range of 100 nm to 20 μM. Table 3 provides the inhibitory concentration of the compounds that results in 50% of the maximal cell count in no treatment conditions (IC₅₀ values; a number not relative to the initial cell count). FIG. 20 shows the dose response curves for the tested compounds and combinations. All conditions resulted in near 0% cell viability suggestive of antiproliferative and cell death capability in the concentration range examined. DefNEtTrp demonstrated a superior cytotoxic effect than Def, NEtTrp, and Trp in single drug and combination drug treatments.

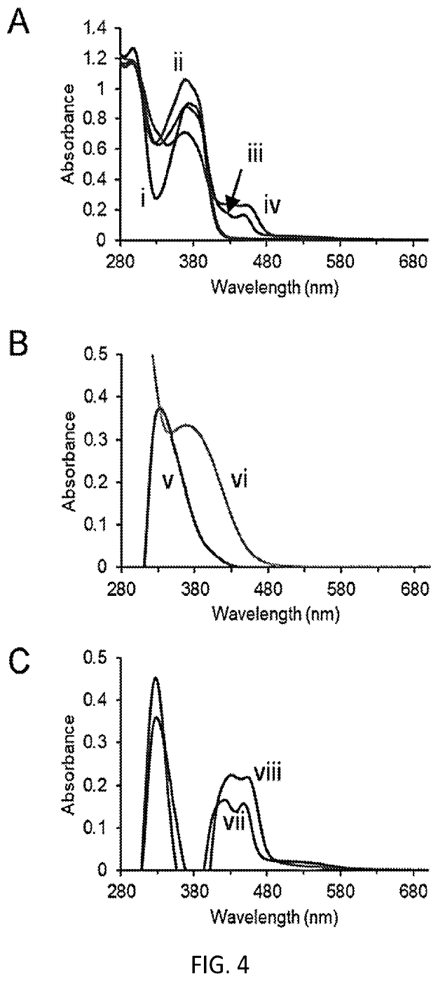

The heightened potency of DefNEtTrp is believed to be owed to conjugation of the two chelators facilitating a synergistic redox activity (see FIGS. 4A-4C). As already established, the Fe(III) coordination to the Trp moiety would be able to redox cycle and reduce the RNR R2 tyrosyl radical and inactivate the enzyme. In a previous study, it was reported that redox active Fe(III) monoDef species could potentially form intracellularly (Gaur et al., 2021, Iron chelator transmetalative approach to inhibit human ribonucleotide reductase. JACS Au 1 (6): 865-878) These species may be expected to readily reduce to Fe(II), release the Fe(II), and redox cycle by binding and reducing other Fe(III) ions. Thus the dual chelator DefNEtTrp could result in simultaneous formation of Fe(monoDef) and Fe(monoTrp) species, producing a powerful reductant particularly of the RNR R2 tyrosyl radical and potentially generate uncontrolled ROS formation.

TABLE 3
IC₅₀ values of the compounds Trp, NEtTrp, Def, DefNEtTrp,
1:1 combination of Def and NEtTrp and 1:1 combination
of Def and Trp treated against Jurkat cell line for 72 h.

| Compounds | IC₅₀ (μM) |
|---|---|
| Trp | 1.1 ± 0.04 |
| NEtTrp | 5.9 ± 0.29 |
| Def | 2.6 ± 0.15 |
| DefNEtTrp | 0.77 ± 0.06 |
| 1:1 combination of | 4.9 ± 0.18 |
| Def and NEtTrp | |
| 1:1 combination of | 3.1 ± 0.17 |
| Def and Trp | |

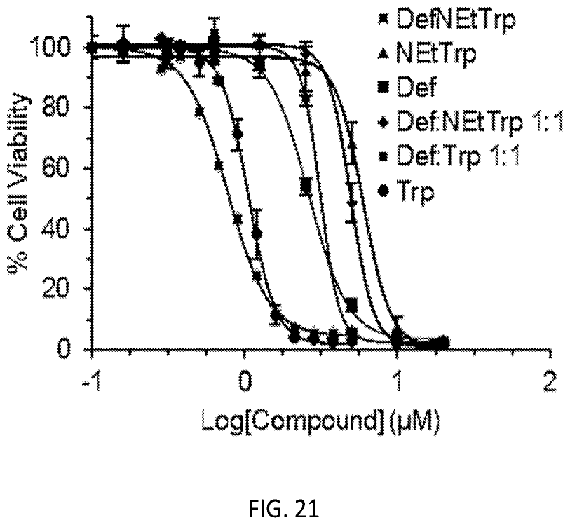

An additional cell viability experiment was performed to examine the effect that supplementation of Fe(III) to Jurkat cells would have if performed prior to treatment with a cytotoxic dose of DefNEtTrp. Jurkat cells were supplemented with a nontoxic 2 μM and 5 μM dose of Fe(III) in the form of in situ Fe(Citrate)₂ two hours before addition of 2 μM DefNEtTrp, respectively. After 72 h treatment with DefNEtTrp, the cells that were supplemented with Fe(III) exhibited a significant increase in viability relative to the non-supplemented group (see FIG. 21). These findings were consistent with expectations because the Fe-binding capability of the ligand would be blocked especially at the higher dosage of supplemented Fe(III). Furthermore, previous cell viability studies with Fe(III)-bound Def and Fe(III)-bound Trp demonstrated that the metal compounds were significantly less cytotoxic than the metal-free ligand.

The cancer selectivity of DefNEtTrp. Given its broad spectrum and potent cytotoxicity against different cancer cells, DefNEtTrp was evaluated against two human noncancer cells: MRC-5 lung cells (see FIG. 22) and red blood cells (see FIG. 26). The compound demonstrated no antiproliferative behavior against MRC-5 in the concentration range examined (maximum concentration of 100 μM). Up to a concentration of 50.3 μM, the compound induced minor hemolysis (˜5%) comparable to the behavior of its constituent groups triapine, NEtTrp, and Def. These concentrations were well above the GI₅₀/IC₅₀ and TGI values of DefNEtTrp against all of the cancer cells examined in this work, indicative of the compound being highly selective for cancer cells.

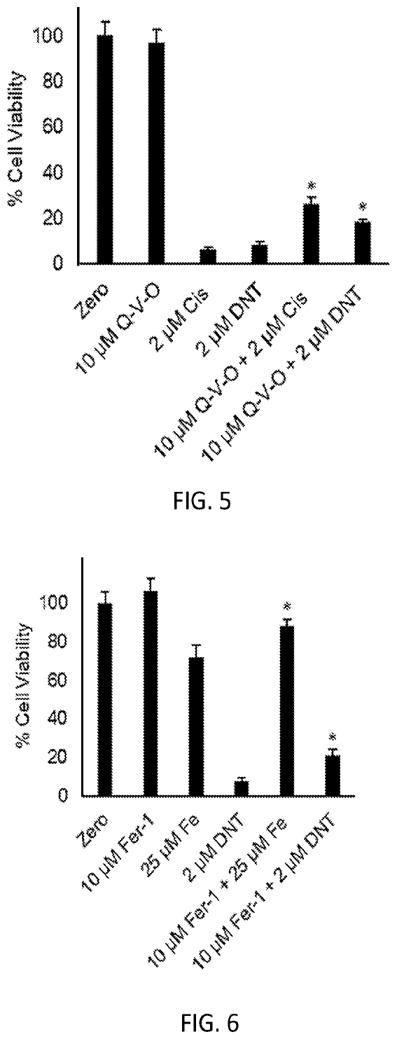

Cell death pathways induced by DefNEtTrp. Previous studies have demonstrated that the Def ligand can induce apoptotic cell death whereas the Trp ligand can induce both apoptosis and ferroptosis (Loza-Rosas et al., 2017, Expanding the therapeutic potential of the iron chelator deferasirox in the development of aqueous stable Ti(IV) anticancer complexes. Inorg. Chem. 56 (14): 7788-7802). With this in mind, the apoptotic and ferroptotic capability of DefNEtTrp was examined in Jurkat Cells using the apoptosis inhibitor Q-VD-OPh and the ferroptosis inhibitor ferrostatin-1 (Fer-1). Jurkat cells pretreated with Q-VD-OPh (10 μM) experienced a two-fold increase in viability against treatment with 2 μM DefNEtTrp, which suggests that the dual chelator can induce apoptosis (see FIGS. 4A-4C). This result compared favorably with the finding that the apoptosis-inducing cisplatin (2 μM) experiences less antiproliferative activity against Jurkat cells when pretreated with the inhibitor. Work with the ferroptosis inhibitor, Fer-1, also revealed that DefNEtTrp triggers ferroptosis cell death given the two-fold increase in the cell viability of Jurkat cells pretreated with the inhibitor (10 μM) in the presence of 2 μM DefNEtTrp (FIG. 5). This behavior was supported by the positive control Fe(citrate)₂ at a relatively high concentration (20 μM) (Wu et al., 2021, Ammonium Ferric Citrate induced Ferroptosis in Non-Small-Cell Lung Carcinoma through the inhibition of GPX4-GSS/GSR-GGT axis activity. Int. J. Med. Sci. 18 (8): 1899-1909). A co-treatment experiment was performed with equimolar Q-VD-OPh and Fer-1, which resulted in a three-fold increase in the viability of Jurkat cells against treatment with 2 μM (see FIG. 23).

### CONCLUSION

The results set forth herein demonstrated the rich anticancer potential of the dual chelator ligand DefNEtTrp. Designed to operate by reaction with the LIP of the intracellular environment, it retained the Fe(II) and Fe(III) binding capacity of its constituent chelating moieties and readily reacted with labile Fe(III) without scavenging the metal ion from sTf. The NCI60 cancer screen of DefNEtTrp revealed a potent antiproliferative broad spectrum cytotoxic profile specific to cancer cells. A comparative study of the cytotoxicity of the dual chelator ligand versus that of the unconjugated chelators in Jurkat cells demonstrated that not only is the antiproliferative behavior of DefNEtTrp superior but also its conjugation appears to facilitate a synergism that may be owed to the redox activity of Fe coordination to both chelator moieties (see FIG. 6). Fe(ILI) binding at the Trp moiety, at minimum, exhibited a reduction potential well within the biological window to be an effective reductant of the RNR R2 tyrosyl radical and thus inactivate the enzyme and also redox cycle. In a previous study, it was reported that redox active Fe(Ill) monoDef species could potentially form intracellularly (Gaur, 2021, Id.). These species can be expected to readily reduce to Fe(II), release the Fe(II), and redox cycle by binding and reducing other Fe(III) ions. Thus the dual chelator DefNEtTrp could result in simultaneous formation of Fe(monoDef) and Fe(monoTrp) species, producing a powerful reductant particularly of the RNR R2 tyrosyl radical and could potentially generate uncontrolled ROS formation.

The cell death capability of DefNEtTrp depends on its Fe-binding ability as demonstrated by cells pre-supplemented with Fe demonstrating higher resistance toward the cytotoxicity of the dual chelator. The compound induced both apoptotic and ferroptotic cell death pathways. These findings attested to the importance of Fe chelation in its mechanism of action and also to the promise of metalation enriching the use of the ligand. On this note, the successful synthesis of heterometal compounds bodes well for future studies on synergizing cytotoxic metals with the ligand. Studies by Contel et al. have demonstrated a profound synergism in attacking various cancer cell lines with heterometal compounds that could not have been achieved by treatment with the individual metal ions and the ligands as separate compounds (Fernandez-Gallardo et al., 2014, Organometallic titanocene-gold compounds as potential chemotherapeutics in renal cancer. Study of their protein kinase inhibitory properties. Organometallics 33 (22): δ669-6681; Femindez-Gallardo et al., 2015, Heterometallic titanium-gold complexes inhibit renal cancer cells in vitro and in vivo. Chemical Science 6 (9): 5269-5283; Elie et al., 2019, Bimetallic titanocene-gold phosphane complexes inhibit invasion, metastasis, and angiogenesis-associated signaling molecules in renal cancer. Eur. J. Med. Chem. 161: 310-322; Massai et al., 2015, Design, synthesis and characterisation of new chimeric ruthenium (II)-gold (I) complexes as improved cytotoxic agents. Dalton Trans. 44 (24): 11067-11076; Elie et al., 2019, Bimetallic titanocene-gold phosphane complexes inhibit invasion, metastasis, and angiogenesis-associated signaling molecules in renal cancer. Eur. J. Med. Chen. 161: 310-322). In sum, simple conjugation of two chelator moieties with soft and hard Lewis basicity elucidated a new direction in the use of coordination chemistry for the clever manipulation of labile cellular physiological metals for therapeutic effect.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features.

1. A metal chelation compound, the compound having the structure:
D-L-T, wherein
D is deferasirox or a derivative thereof,
T is triapine or a derivative thereof, and
L is a linking group and is a bond, C₁-C₁₀ alkyl, C₂-C₁₀ alkenyl, or —(C₀-C₃ alkyl)-R—(C₀-C₃ alkyl)-,
wherein R is aryl, cycloalkyl, or polyethylene glycol.

2. The metal chelation compound of claim 1, wherein D is an amide derivative of deferasirox, 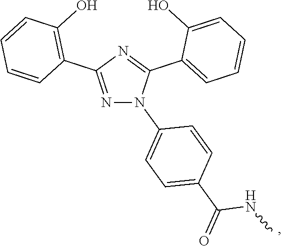
or a salt thereof.

3. The metal chelation compound of claim 1, wherein T is 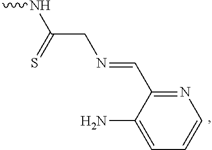
or a salt thereof.

4. The metal chelation compound of claim 1, wherein L is C₁-C₈ alkyl, C₂-C₈ alkenyl, or —(C₀-C₃ alkyl)-R—(C₀-C₃ alkyl)-, wherein R is phenyl, or polyethylene glycol, wherein the polyethylene glycol comprises 1 to 5 ethylene units.

5. The metal chelation compound of claim 1, wherein L is a C₁-C₄ alkyl group.

6. The metal chelation compound of claim 5, wherein L is —CH₂—CH₂—.

7. The metal chelation compound of claim 1, wherein the compound is 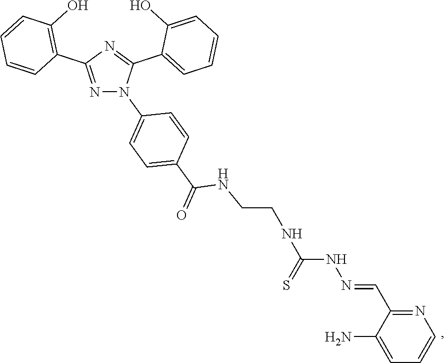 [SMILES: Nc1cccnc1/C=N/NC(=S)NCCNC(=O)c1ccc(-n2nc(-c3ccccc3O)nc2-c2ccccc2O)cc1]
or a salt thereof.

8. A coordination complex, wherein the coordination complex comprises at least one of the metal chelation compound of claim 1 coordinated to at least one metal ion, wherein each metal ion is independently iron, titanium, platinum, vanadium, copper, or gold.

9. The coordination complex of claim 8, comprising two metal chelation compounds that come together to coordinate one metal ion.

10. The coordination complex of claim 8, wherein the complex has the structure (T-L-D)-M-(D-L-T), M-(T-L-D)-M-(D-L-T), or M-(T-L-D)-M-(D-L-T)-M, wherein M is a metal ion, wherein each M is independently iron, titanium, platinum, vanadium, copper, or gold.

11. (canceled)

12. The coordination complex of claim 10, wherein the -D-M-D- moiety forms a six-coordinate sphere around the central M.

13. The coordination complex of claim 8, wherein each metal ion is titanium or iron.

14. (canceled)

15. The coordination complex of claim 13, wherein each metal ion is Fe(Ill).

16. A method of preparing a metal coordination compound, the method comprising:
providing a triapine derivative of structure: 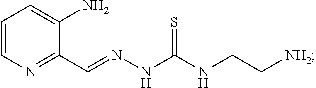 [SMILES: NCCNC(=S)N/N=C/c1ncccc1N]
contacting the triapine derivative with deferasirox in an anhydrous polar solvent in the presence of base and hydrobenzotriazole to form a crude mixture;
contacting the crude mixture with acid to form a precipitate; and
isolating the precipitate.

17. (canceled)

18. A method for preparing a coordination complex, the method comprising:
providing the metal chelation compound of claim 1, and
contacting the metal chelation compound with a metal salt in a solvent, wherein the metal of the metal salt is iron, platinum, titanium, vanadium, copper, or gold.

19. A method for treating cancer in a subject in need thereof, the method comprising administering a therapeutically effective amount of the compound of claim 1 or a coordination complex thereof, wherein the compound of claim 1 is coordinated to at least one metal ion, wherein each metal ion is independently iron, titanium, platinum, vanadium, copper, or gold.

20. The method of claim 19, wherein the cancer is leukemia, lung cancer, colon cancer, CNS cancer, melanoma, ovarian cancer, renal caner, prostate cancer, or breast cancer.

21. The method of claim 20, wherein the lung cancer is a non-small cell lung cancer.

22. The method of claim 19, wherein the compound or coordination complex is administered in an amount in the range of 0.1 mg/kg to 400 mg/kg.

23. The method of claim 19, wherein the administering results in an antiproliferative effect against non-cancerous cells of less than 20%.

24-25. (canceled)
